(19) United States
(12) Patent Application Publication
(10) Pub. No.: US 2008/0300202 A1
(43) Pub. Date: Dec. 4, 2008

(54) SUBTRACTIVE TRANSGENICS

(75) Inventor: Clifford Kentros, Eugene, OR (US)

Correspondence Address: KLARQUIST SPARKMAN, LLP, 121 SW SALMON STREET, SUITE 1600, PORTLAND, OR, 97204, US

(73) Assignees: The State of Oregon acting by and through the State Board of Higher Education on behalf of the; University of Oregon

(21) Appl. No.: 11/750,971

(22) Filed: May 18, 2007

Related U.S. Application Data
(60) Provisional application No. 60/801,692, filed on May 18, 2006.

(51) Int. Cl. A61K 31/70; A01K 67/027; C12N 15/00; A61P 43/00; C12N 5/06

(52) U.S. Cl. 514/44; 800/13; 435/325; 800/22; 435/320.1

(57) ABSTRACT

Described herein are methods for predictable, highly selective expression of a transgene in a human or non-human animal. The methods comprise subtractive transgenics, wherein the expression pattern of one selective promoter is subtracted from the expression pattern of a second selective promoter. Provided are non-human transgenic animals exhibiting a highly selective expression pattern and methods of producing such animals. Further provided are methods of selective expression of a transgene in an animal, including a human, by administration of viral vectors.

### CROSS REFERENCE TO RELATED APPLICATION

This application claims the benefit of priority of U.S. Provisional Application Ser. No. 60/801,692, filed May 18, 2006, which is herein incorporated by reference.

### STATEMENT OF GOVERNMENT SUPPORT

This invention was made with United States government support pursuant to grant #DAMD17-01-1-0750, from the Department of Defense; the United States government has certain rights in the invention.

### FIELD

This disclosure relates to a method for predictable, highly selective expression of a transgene in a human or non-human animal. The disclosure further relates to non-human transgenic animals exhibiting a highly selective expression pattern and methods of producing such animals.

### BACKGROUND

Selective expression of a gene or transgene in particular cell populations is valuable for a number of research and therapeutic applications. The cloning and use of native promoters to direct expression of a gene in a selected cell type has been well described. However, native promoters often do not provide the level of selectivity required.

Previous studies have described conditional and inducible transgene expression through the combined use of Cre-mediated recombination and the tetracycline-dependent expression system (Belteki et al. Nucl. Acids Res. 33(5):e51, 2005; Ivy Yu et al. Proc. Natl. Acad. Sci. U.S.A. 102(24):8615-8620, 2005). The Cre-loxP system allows for the permanent activation or deactivation of genes in a particular cell lineage. The tetracycline-dependent system permits temporal and cell-type specific control of transgene expression. However, even with the combination of these two systems, transgene expression is only as specific as the promoter used to drive expression of the Cre recombinase.

Thus, provided herein is a method for the predictable, highly selective expression of a transgene using subtractive transgenics. As described herein, subtractive transgenics involves the use of two different selective promoters, wherein the expression pattern of one promoter is subtracted from the expression pattern of a second promoter to enable specificity of transgene expression in a sub-population of cells.

### SUMMARY

Described herein are methods of subtractive transgenics, wherein the expression pattern of one selective promoter is subtracted from the expression pattern of a second selective promoter. Provided herein is a transgenic non-human animal comprising (a) a first heterologous nucleic acid encoding a recombinase operably linked to a first selective promoter; (b) a second heterologous nucleic acid encoding a transcription factor operably linked to a second selective promoter, wherein expression of the transcription factor is regulated by expression of the recombinase; and (c) a third heterologous nucleic acid comprising a transgene under the transcriptional control of a response element specific for the transcription factor. In one embodiment, in the transgenic non-human animal the recombinase is expressed in a first population of cells and the transcription factor is expressed in a second population of cells, wherein the first and second populations of cells comprise at least one overlapping sub-population of cells and at least one non-overlapping sub-population of cells. In one embodiment, the first and/or second selective promoter comprises a tissue specific promoter. In another embodiment, the first and/or second selective promoter comprises a temporally specific promoter. In another embodiment, the first and/or second selective promoter comprises an inducible promoter.

Further provided is a method of producing a non-human animal that expresses a transgene in a selected population, or sub-population of cells, the method comprising identifying at least one progeny of a cross between (a) a first transgenic non-human animal comprising a first heterologous nucleic acid encoding a recombinase operably linked to a first selective promoter; (b) a second transgenic non-human animal comprising a second heterologous nucleic acid comprising a polynucleotide sequence that encodes a transcription factor operably linked to a second selective promoter, wherein the polynucleotide sequence is (i) flanked by recombinase recognition sites; or (ii) preceded by a transcription STOP signal flanked by recombinase recognition sites, wherein the at least one identified progeny comprises the first and second heterologous nucleic acids and wherein the at least one progeny further comprises a transgene under the transcription regulatory control of a response element specific for the transcription factor.

Also provided is a method for selective expression of a transgene in a population, or sub-population, of cells in an animal, comprising administering to the animal (a) a first viral vector comprising a nucleic acid encoding a recombinase operably linked to a nucleic acid encoding a first selective promoter; (b) a second viral vector comprising a nucleic acid encoding a transcription factor operably linked to a nucleic acid encoding a second selective promoter, wherein expression of the transcription factor is regulated by expression of the recombinase; and (c) a third viral vector comprising a transgene under the transcriptional control of a response element specific for the transcription factor. In some embodiments, the first, second and third viral vectors are independently selected from adenovirus vectors, adeno-associated virus vectors, lentivirus vectors, retrovirus vectors and herpesvirus vectors. In one embodiment, the transgene is a therapeutic molecule, a toxin or a maker protein.

Also provided herein are kits for subtractive transgenics. In one embodiment, the kit comprises (1) a first viral vector comprising a nucleic acid encoding a recombinase operably linked to a nucleic acid encoding a first selective promoter; (2) a second viral vector comprising a nucleic acid encoding a transcription factor operably linked to a nucleic acid encoding a second selective promoter; and (3) a third viral vector comprising a transgene under the transcriptional control of a response element specific for the transcription factor. In another embodiment, the kit comprises (1) a first injection construct comprising a nucleic acid encoding a recombinase operably linked to a first selective promoter; (2) a second injection construct comprising a nucleic acid encoding a transcription factor operably linked to a second selective promoter, wherein expression of the transcription factor is regulated by expression of the recombinase; and (3) a third injection construct comprising a transgene under the transcriptional control of a response element specific for the transcription factor.

The foregoing and other features and advantages will become more apparent from the following detailed description of several embodiments, which proceeds with reference to the accompanying figures.

### BRIEF DESCRIPTION OF THE FIGURES

FIG. 1A schematically illustrates tissue-specific knock-out of a target gene by Cre recombinase. A tissue-specific promoter (Promoter X) driving expression of the Cre recombinase mediates excision of a target gene flanked by loxp recognition sites. FIG. 1B schematically illustrates tissue-specific, inducible (by doxycycline) expression of a target gene under transcriptional control of the tetO operator. In the absence of doxycycline, tTA is capable of binding tetO, driving expression of the transgene. Expression of tTA is regulated by a tissue-specific promoter (Promoter X).

FIG. 2 schematically illustrates an example of “subtractive transgenics.” In this example, promoter “ABCD” drives expression of tTA in cell types A, B, C and D. Promoter “BCD” drives expression of Cre in cell types B, C and D. Where expression overlaps (cell types B, C and D), Cre will excise tTA through recognition of the flanking loxp sites. Therefore, tTA will be expressed only in cell type A.

FIG. 3 schematically illustrates the expression patterns of a transgene in subtractive transgenic animals using the ftTA (A) and fSTOP-tTA (B) strategies.

FIG. 4 schematically illustrates the CamKIIα-ftTA construct (pMM403 backbone).

FIGS. 5A-C are images of in situ staining of subsets of neurons in strains of mice expressing tTA (A and C) and Cre (B and C) under the transcriptional control of the CamKIIα promoter. When both tTA and Cre are under the control of the same promoter (C), tTA (flanked by loxp sites) is not expressed.

FIG. 6 schematically illustrates a strategy for creating ftTA BAC vectors.

FIGS. 7A and B schematically illustrate transgenic BAC generation and selective expression, respectively.

FIGS. 8A and B schematically illustrate the strategy for PCR screening of native and recombinant BAC for Drd4 promoter constructs. For PCR specific for native BAC (FIG. 8A), both the forward primer (Drd4_{—}5′i) and reverse primer (Drd_gene_rev) are in the BAC vector. For PCR specific for recombinant BAC (FIG. 8B), the forward primer (Drd4_{—}5′i) is in the BAC and the reverse primer (pLD800_rev) is in the insert from the shuttle vector.

FIGS. 9A and B schematically illustrate the strategy for Southern blot identification of co-integrants. The Drd4_{—}5′ probe binds to a part of the BAC upstream of the A box. Both the native and recombinant BACs are subjected to XbaI digestion, which cuts the BAC at a point upstream of the binding site for the probe, and at a site that is downstream of the end point of the A box. The distance between the two sites is approximately 7.1 kb. If tTA is inserted homologously with the A boxes synchronizing between the BAC and the shuttle vector, then an extra XbaI site is introduced since it is present in the ftTA cassette, and the fragment that binds to the probe is shortened to 3.4 kb. The tTA probe binds to the ftTA inset from the recombination cassette, and therefore only to the recombinant BAC. In the native BAC (FIG. 9A), the Drd4_{—}5′ probe binds to the >7 kb band and the tTA will not bind. In the recombinant BAC (FIG. 9B), Drd4_{—}5′ will bind to the >3 kb band and tTA will bind to a band of variable size depending on the size of the insert recombined (>5 kb if at least part of the DNA up to the β-lactamase gene conferring Amp resistance has been inserted).

FIGS. 10A-C are images demonstrating hybridization of BAC DNA for characterization of recombination of the Drd4 constructs. FIG. 10A shows RNA-based screening of 40 potential colonies for recombination. All clones were identified as positive. FIG. 10B is an image of DIG-labeled screening of RP23-134L4-based recombination, hybridized with tTA (left) and with Drd4_{—}5′ (right). The left lane in both panels is the recombinant (R) and the right lane is the control (C). FIG. 10C is an image of DIG-labeled screening of sixteen colonies with Drd4_{—}5′, which shows recombination in all eleven RP23-320N24 colonies (˜3.4 kb band) and no recombination in any of the RP23-134L4 colonies (˜7.1 kb band).

FIGS. 11A-C illustrate the strategy for Southern blot identification of co-integrants for the ChAT promoter constructs. The ChAT_Abox probe binds to the A box. Both the native and recombinant BACs are subjected to XbaI digest, which cuts the BAC at a point upstream of the binding site for the probe, and at a site that is downstream of the end point of the A box. In the native BAC RP23-246B12 (FIG. 11A), the distance between the two sites is approximately 5.8 kb. If tTA is inserted homologously with the A boxes synchronizing between the BAC and the shuttle vector, an additional XbaI site is introduced since it is present in the ftTA cassette. Thus, the fragment binding to the probe is shortened to approximately 4.3 kb. In the recombinant BAC (FIG. 11B), the ChAT_Abox probe binds to the (approximately) 4.3 kb band and also to the (approximately) 6.2 kb band as the whole shuttle vector sequence is repeated in reverse. The recombinant shows binding of probe to 4.3 kb and 6.2 kb bands, as opposed to 5.8 kb for the native BAC. Drd4 ftTA does not bind to the probe since it doesn't contain the ChAT A box sequence (FIG. 11C).

FIG. 12 is a map of the pBS_CALB_tTA injection construct (SEQ ID NO: 24).

FIG. 13 is a map of the pBS_CALB_fSTOPtTA injection construct (SEQ ID NO: 25).

FIG. 14 is a map of the pBS_CCK-fTA injection construct (SEQ ID NO: 26).

FIG. 15 is a map of the pBS_CCK-fSTOPtTA injection construct (SEQ ID NO: 27).

FIGS. 16A-E are images of immunohistochemical staining of hippocampal sections from transgenic mouse lines expressing tTA and/or Cre under the transcriptional control of CamKIIα. The genotype of each mouse is shown in the upper left of each image. FIG. 16A is a hippocampal section from an unsubtracted mouse (ftTA⁺/Cre⁻) showing detection of tTA expression in all primary neurons (CA1, CA3, gc and hilus). FIG. 16B is a hippocampal section from a subtracted mouse (ftTA⁺/Cre⁺) in which Cre is expressed only in CA3 neurons. In the same subtracted mouse, tTA expression is observed in CA1, gc and hilus (all primary neurons except CA3). FIG. 16D and FIG. 16E are higher magnification images of tTA expression in the CA3/hilar area in the unsubtracted line and the subtracted line, respectively. The caret indicates the CA2/CA3 boundary. tTA expression was detected using an anti-tTA primary antibody (VP16, Chemicon). Cre expression was detected using an anti-Cre primary antibody (Novagen). An HRP-linked (Chemicon) secondary antibody was used to catalyze the diaminobenzidine (DAB) reaction.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is the nucleotide sequence of primer DrdABox5′Asc.

SEQ ID NO: 2 is the nucleotide sequence of primer DrdABox3′Fse.

SEQ ID NO: 3 is the nucleotide sequence of the loxP1 oligonucleotide.

SEQ ID NO: 4 is the nucleotide sequence of the loxP1bis oligonucleotide.

SEQ ID NO: 5 is the nucleotide sequence of the loxP2 oligonucleotide.

SEQ ID NO: 6 is the nucleotide sequence of the loxP2bis oligonucleotide.

SEQ ID NO: 7 is the nucleotide sequence of primer T3.

SEQ ID NO: 8 is the nucleotide sequence of primer T7.

SEQ ID NO: 9 is the nucleotide sequence of primer RecA_{—}7023fwd.

SEQ ID NO: 10 is the nucleotide sequence of primer EGFP_{—}150rev.

SEQ ID NO: 11 is the nucleotide sequence of primer Drd4_{—}5′i.

SEQ ID NO: 12 is the nucleotide sequence of primer Drd4_gene_rev.

SEQ ID NO: 13 is the nucleotide sequence of primer pLD800_rev.

SEQ ID NOs: 14 and 15 are the nucleotide sequences of primers used to amplify segment A of the CCK promoter.

SEQ ID NOs: 16 and 17 are the nucleotide sequences of primers used to amplify segment B of the CCK promoter.

SEQ ID NOs: 18 and 19 are the nucleotide sequences of primers used to amplify segment C of the CCK promoter.

SEQ ID NOs: 20 and 21 are the nucleotide sequences of oligonucleotides used to modify the multiple cloning site of pBSKS+.

SEQ ID NO: 22 is the nucleotide sequence of the ChAT_ftTA injection construct.

SEQ ID NO: 23 is the nucleotide sequence of the Drd4_ftTA injection construct.

SEQ ID NO: 24 is the nucleotide sequence of the pBS_CALB_ftTA injection construct.

SEQ ID NO: 25 is the nucleotide sequence of the pBS_CALB_fSTOPtTA injection construct.

SEQ ID NO: 26 is the nucleotide sequence of the pBS_CCK_ftTA injection construct.

SEQ ID NO: 27 is the nucleotide sequence of the pBS_CCK_fSTOPtTA injection construct.

### DETAILED DESCRIPTION

### I. Abbreviations

AAV Adeno-associated virus

APP Amyloid precursor protein

BAC Bacterial artificial chromosome

CamKII Calcium-calmodulin-dependent protein kinase II

CBP Calcium binding protein

CCK Cholecystokinin

CD Cluster of differentiation

CHAT Choline acetyltransferase

CNS Central nervous system

CSD Current source density

DRD4 Dopamine receptor D4

ES Embryonic stem

GENSAT Gene Expression Nervous System Atlas

GFP Green fluorescent protein

HIV Human immunodeficiency virus

IRES Internal ribosomal entry site

NPY Neuropeptide Y

PFC Prefrontal cortex

SMT Somatostatin

tetO Tetracycline operator

VIP Vasoactive intestinal peptide

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Animal: Refers to a human or non-human organism. A non-human animal can be a vertebrate such as a fish, an amphibian, a reptile, a bird or a mammal. In some cases, the non-human animal is a rodent, such as a mouse. In other cases, the non-human animal is a domesticated livestock, such as a cow, a sheep, a goat, a pig or a horse. In other cases, the non-human animal is a non-human primate.

Apoptosis: A type of cell death in which a cell uses specialized cellular machinery to kill itself. Apoptosis is a cellular suicide mechanism which occurs during development and in response to specific external stimuli, such as infection or injury. As used herein, a “pro-apoptotic molecule” (such as a pro-apoptotic gene or protein) is a molecule that when expressed, promotes, enhances or increases apoptosis in a cell, or increases susceptibility of a cell to apoptosis. Pro-apoptotic molecules are well known in the art and include, for example, caspases, granzyme B, Fas/Fas ligand, Bcl-2 and Bcl-X (and related family members), ceramide, smac, tumor necrosis factor and death domain containing molecules.

Enabled: As used herein, “enabled” means made possible. Thus, expression is enabled by a molecule, event or occurrence, if the presence of the molecule, or the occurrence of the event makes expression possible. In contrast, the term “disabled” means to make incapable. Thus, expression is disabled by a molecule, event or occurrence, if the presence of the molecule or occurrence of the event eliminates or drastically reduces the capacity for expression.

Embryonic stem (ES) cells: Pluripotent cells isolated from the inner cell mass of the developing blastocyst. ES cells are pluripotent cells, meaning that they can generate all of the cells present in the body (bone, muscle, brain cells, etc.). Methods for producing murine ES cells can be found in U.S. Pat. No. 5,670,372, herein incorporated by reference. Methods for producing human ES cells can be found in U.S. Pat. No. 6,090,622, PCT Publication No. WO 00/70021 and PCT Publication No. WO 00/27995, herein incorporated by reference.

Excision: As used herein, “excision” means removal.

Expression: The transcription and/or translation of a product from a nucleic acid template. Thus, an organism “expresses” a transgene when at least a portion of the heterologous nucleic acid is transcribed and, in some instances, translated.

Expression control sequence: Includes nucleic acid sequences that regulate the expression of a nucleic acid sequence to which it is operably linked. A nucleic acid is operably linked to an expression control sequences when the expression control sequences control and regulate the transcription and, as appropriate, translation of the nucleic acid sequence. Thus, expression control sequences can include promoters, enhancers, transcription terminators, a start codon (typically, ATG) in front of a protein-encoding gene, splicing signal for introns, maintenance of the correct reading frame of that gene to permit proper translation of mRNA, and stop codons.

Expression vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes.

Flanks: As used herein, a first nucleotide sequence “flanks” a second nucleotide sequence if copies of the first nucleotide sequence are present (in any orientation or position) on both sides of the second nucleotide sequence. In which case, the second nucleotide sequence is said to be “flanked” by the first nucleotide sequence. In the context of this disclosure, for example, a polynucleotide sequence is flanked by a recognition site for a site-specific recombinase if a recognition site is present on both sides of the polynucleotide sequence.

Floxed: Refers to a nucleic acid that is flanked by loxP sites, which are recognition sites for Cre recombinase. In one embodiment, the “floxed” nucleic acid is a transactivator or transcription factor, such as tTA. In another embodiment, the “floxed” nucleic acid is a STOP signal.

Heterologous nucleic acid: A nucleic acid that does not originate in a particular cell or organism in nature. For example, a heterologous nucleic acid can be a nucleic acid that includes a polynucleotide sequence of a different organism or a recombinant, synthetic or artificial polynucleotide sequence. In the context of this disclosure a “transgene” is a heterologous nucleic acid.

Infective: A virus or vector is “infective” when it transduces a cell, replicates, and (without the benefit of any complementary virus or vector) spreads progeny vectors or viruses of the same type as the original transducing virus or vector to other cells in an organism or cell culture, where the progeny vectors or viruses have the same ability to reproduce and spread throughout the organism or cell culture.

Injection construct: A nucleic acid sequence suitable for injection into an oocyte, embryonic stem cell, or other appropriate cell type, for homologous recombination and/or the generation of a transgenic animal. In one embodiment, the injection construct comprises a heterologous nucleic acid, such as a nucleic acid encoding a recombinase or a transcription factor, and selective promoter.

Neural cell: Any cell in a lineage that originates with a neural stem cell and includes a mature neuron. Thus, the term neural cell includes neurons (nerve cells) as well as their progenitors regardless of their stage of differentiation. In the context of an adult brain, neural cells are predominantly differentiated neurons. In contrast, a “non-neural cell” is a cell of a lineage other than a neural cell lineage, that is, a lineage that does not culminate in the differentiation of a mature neuron. The non-neural cell may reside in the central nervous system (CNS), for example, in the brain (such as glial cells and immune system cells, such as B cells, dendritic cells, macrophages and microglia), or may exist in an organ outside the CNS, such as cardiac, skeletal or smooth muscle (a muscle cell), liver (a hepatic cell) or kidney (a renal cell) and so forth. Non-neural cells include cells of the immune system, regardless of whether they reside in the CNS or elsewhere in the body of the organism.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically Acceptable Vehicle: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds or molecules, such as one or more viral vectors and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating: “Preventing” a disease or condition refers to inhibiting the full development of a disease or condition. “Treating” refers to a therapeutic intervention that ameliorates a sign or symptom of a disease or pathological condition after it has begun to develop. “Ameliorating” refers to the reduction in the number or severity of signs or symptoms, or the duration, of a disease or condition.

Population: As used herein, a “population” or “set” of cells refers to any identifiable plurality of cells. A population of cells can include one or more cell types, and/or can include functionally or spatially related cells or cells that are related by lineage or involvement in a cellular pathway. A “sub-population” or “subset” of cells refers to an identifiable group of cells (or cell types) within a population. An “overlapping” sub-population of cells is a group of cells (or cell types) that share at least one common feature between two or more populations of cells.

Promoter: A sequence sufficient to direct transcription, and which may optionally include additional polynucleotide sequences. In some cases the promoter is a selective promoter capable of rendering promoter-dependent gene expression, for instance which is selective for a specific cell-type, a specific tissue, or a specific time point during development or differentiation. Selective promoters can also be inducible by external signals or agents (that is, “inducing agents”). Selective promoters can modulate anatomical, cell, tissue, temporal and/or spatial expression of a nucleic acid, such as a transgene.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Sambrook et al. (ed.), Molecular Cloning: A Laboratory Manual, 2^{nd} ed., vol. 1-3, Cold Spring Harbor Laboratory Press, Cold Spring Harbor, N.Y., 1989. The term recombinant includes nucleic acids that have been altered by addition, substitution, or deletion of a portion of the nucleic acid.

Recombinase: Any polypeptide that mediates in whole or in part the process of nucleic acid recombination. A “site specific” recombinase is a recombinase that mediates recombination of a nucleic acid that possesses particular nucleotide binding sites, termed “recognition sites.” Examples of site specific recombinases and their corresponding recognition sites include the Cre recombinase and corresponding loxP recognition sites, flp recombinase and corresponding FRT recognition sites, and the bacterial β-recombinases and their corresponding recognition sites.

Silencer: As used herein, a silencer construct is a construct that “turns off,” or inhibits the activity of cells, such as toxins, dominant negative constructs, and inhibitory RNA molecules. Generally, silencers are capable of reducing a cell's response to a stimulus or stimuli.

Specified: As used herein, “specified” with respect to a cell indicates that the cell is of an identifiable lineage and/or phenotype and/or population that can be determined with reliability by a practitioner of ordinary skill in the art.

Subtractive transgenics: Describes methods wherein predictable, highly selective expression of a transgene can be achieved by subtracting the expression pattern of one promoter from the expression pattern of a different promoter.

Target nucleic acid: As used herein, a “target nucleic acid” is a heterologous nucleic acid with a functional attribute of interest that is subject to inducible transcriptional control by one or more specified control factors. Also referred to herein as a “transgene.”

Therapeutic molecule: As used herein, a “therapeutic molecule” (such as a therapeutic gene or protein) can be any molecule that prevents, treats or ameliorates a disease, condition or disorder (such as a genetic disorder, infection or cancer) when expressed in a selected population or sub-population of cells. In one embodiment, a therapeutic molecule is a wild-type version of a mutated gene or protein involved in a particular disease or condition. In another embodiment, the therapeutic molecule is a pro-apoptotic molecule. A pro-apoptotic molecule would be therapeutic, for example, for the treatment of a condition in which cell death is the goal (such as cancer).

Toxin: A poisonous substance produced by living cells or organisms. Toxins are usually proteins that are capable of causing disease by interacting with biological macromolecules such as enzymes or cellular receptors. Toxins vary greatly in their severity, ranging from usually minor and acute to almost immediately deadly (as in botulinum toxin). Biotoxins vary greatly in purpose and mechanism, and can be highly complex or relatively small proteins.

Transduced and Transformed: A virus or vector “transduces” or “transfects” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. Transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Transcription factor: Also referred to herein as a “transcription activator,” is a polypeptide factor that binds to and induces transcription of a polynucleotide sequence operably linked to a polynucleotide sequence including a nucleotide response element specific for that factor. A response element is specific for a specified transcription factor if the transcription factor specifically binds to the nucleotide sequence of the response element. Commonly, a response element is defined by a consensus sequence encompassing nucleotide variants based on shared nucleotide core sequence.

Transcription STOP signal: A nucleotide sequence that terminates transcription of an operably linked polynucleotide sequence.

Transcription (or expression) is said to be “dependent” on a molecule, event or occurrence if it is regulated either positively or negatively by the molecule, event or occurrence. Also referred to as a “transcription stop signal.”

Transgene: An exogenous or heterologous nucleic acid sequence. A transgene is a gene or genetic material that has been transferred by any of a number of genetic engineering techniques from one organism, vector or DNA sample to another. In some cases, a transgene is incorporated into an animal's (or other organism's) germ line. For example, in higher vertebrates this can be accomplished by injecting the foreign DNA into the nucleus of a fertilized embryo. “Transgene” can also describe any DNA sequence that has been introduced into an organism or vector construct. For example, a transgene can be a heterologous nucleic acid introduced into a viral vector. As described herein, a transgene can comprise any one of a number of molecules (genes or proteins), including, but not limited to, therapeutic molecules, wild-type proteins, toxins, pro-apoptotic proteins and marker proteins.

Transgenic: In reference to an animal or cell, “transgenic” indicates that at least one heterologous nucleic acid, a “transgene,” has been introduced into the genome of the animal (or cell). The transgene can be homologously integrated into the genome of the organism based on sequence specific recombination between the heterologous nucleic acid and polynucleotide sequences endogenous to the organism's genome. Alternatively, the transgene can be randomly integrated into a chromosome of the organism. In some instances, the transgene is maintained within one or more cells of the organism without integration into a chromosome. Methods for producing transgenic animals are well known in the art. For example, methods sufficient to guide one of skill in the art in the production of transgenic animals can be found, for example, in Hogan and Costantini, Manipulating the Mouse Embryo (1994) Cold Spring Harbor Laboratory Press, Cold Spring Harbor.

Vector: A nucleic acid molecule introduced into a host cell, thereby producing a transformed host cell. A vector can include nucleic acid sequences that permit it to replicate in the host cell, such as an origin of replication. A vector can also include one or more selectable marker genes and other genetic elements. An insertional vector is capable of inserting itself into a host nucleic acid. In some embodiments herein, a vector comprises an adenovirus vector, an adeno-associated virus vector, a lentivirus vector, a retrovirus vector or a herpesvirus vector.

### III. Overview of Several Embodiments

Described herein are methods of subtractive transgenics, wherein the expression pattern of one selective promoter is subtracted from the expression pattern of a second selective promoter.

Provided herein is a transgenic non-human animal comprising (a) a first heterologous nucleic acid encoding a recombinase operably linked to a first selective promoter; (b) a second heterologous nucleic acid encoding a transcription factor operably linked to a second selective promoter, wherein expression of the transcription factor is regulated by expression of the recombinase; and (c) a third heterologous nucleic acid comprising a transgene under the transcriptional control of a response element specific for the transcription factor.

Further provided is a method of producing a non-human animal that expresses a transgene in a selected population or sub-population of cells, the method comprising identifying at least one progeny of a cross between (a) a first transgenic non-human animal comprising first heterologous nucleic acid encoding a recombinase operably linked to a first selective promoter; and (b) a second transgenic non-human animal comprising a second heterologous nucleic acid comprising a polynucleotide sequence that encodes a transcription factor operably linked to a second selective promoter, wherein the polynucleotide sequence is (i) flanked by recombinase recognition sites; or (ii) preceded by a transcription STOP signal flanked by recombinase recognition sites, wherein the at least one identified progeny comprises the first and second heterologous nucleic acids and wherein the at least one progeny further comprises a transgene under the transcription regulatory control of a response element specific for the transcription factor.

In one embodiment of the methods, the recombinase is expressed in a first population of cells and the transcription factor is expressed in a second population of cells, wherein the first and second populations of cells comprise at least one overlapping sub-population of cells and at least one non-overlapping sub-population of cells. In one example, the transgene is expressed only in the at least one overlapping sub-population of cells. In another example, the transgene is expressed only in the at least one non-overlapping sub-population of cells.

In one embodiment, the transcription factor is disabled by expression of the recombinase. In one example, the second heterologous nucleic acid comprises recombinase recognition sites flanking the transcription factor, such that expression of the transcription factor is disabled by excision of the transcription factor by the recombinase.

In another embodiment, expression of the transcription factor is enabled by expression of the recombinase. In one example, the second heterologous nucleic acid comprises a transcriptional STOP signal preceding the transcription factor that prevents expression of the transcription factor, wherein the transcriptional STOP signal is flanked by recombinase recognition sites, such that expression of the transcription factor is enabled by excision of the transcriptional STOP signal by the recombinase.

The recombinase can be any recombinase known in the art. For example, the recombinase can be a Cre recombinase, a flp recombinase or a β-recombinase. In addition, the transcription factor can be any transcription factor known in the art. In one embodiment of the methods, the transcription factor is a tTA transcription factor or a fusion protein thereof. When tTA is the transcription factor, the response element can be a tetO transcription response element. In one embodiment, the transcription factor binds to the response element in the absence of an inducing agent. In another embodiment, the transcription factor binds to the response element in the presence of an inducing agent. The appropriate inducing agent is selected based upon which transcription factor and response element are used. For example, if the transcription factor and response element are tTA and tetO, respectively, the inducing agent is selected from tetracycline, doxycycline, or a bioactive derivative thereof.

In one embodiment, at least one of the first and second selective promoters comprises a tissue specific promoter. In another embodiment, at least one of the first and second selective promoters comprises a temporally specific promoter. In another embodiment, at least one of the first and second selective promoters comprises an inducible promoter. In one embodiment, at least one of the first and second selective promoters comprises a promoter that is selective for neural cells. In another embodiment, both the first and second selective promoters are selective for neural cells. In one aspect, the first and second selective promoters are selective for different neural cells. In another aspect, the first and second selective promoters are selective for an overlapping subset of neural cells. In another embodiment, at least one of the first and the second selective promoters are selective for non-neural cells.

In one embodiment, the transgene is expressed in granule cells of the dentate gyrus. In another embodiment, the transgene is expressed in cholinergic neurons. In another embodiment, the transgene is expressed in the prefrontal cortex. In another embodiment, the transgene is expressed in a subset of inhibitory interneurons. In one aspect, the subset of inhibitory interneurons is SMT+ interneurons. In another aspect, the subset of inhibitory interneurons is SMT+/CBP+ interneurons.

In one embodiment, the selective promoter is ChAT. In another embodiment, the selective promoter is Drd4. In another embodiment, the selective promoter is CCK. In another embodiment, the selective promoter is calbindin. In another embodiment, the selective promoter is CamKIIα.

Also provided is an isolated transgenic cell from a transgenic non-human animal described herein.

Further provided is a method for selective expression of a transgene in a population or sub-population of cells in an animal, comprising administering to the animal (a) a first viral vector comprising a nucleic acid encoding a recombinase operably linked to a nucleic acid encoding a first selective promoter; (b) a second viral vector comprising a nucleic acid encoding a transcription factor operably linked to a nucleic acid encoding a second selective promoter, wherein expression of the transcription factor is regulated by expression of the recombinase; and (c) a third viral vector comprising a transgene under the transcriptional control of a response element specific for the transcription factor. In one embodiment, the animal is a non-human animal. In another embodiment, the animal is a human.

In some embodiments, the first, second and third viral vectors are independently selected from adenovirus vectors, adeno-associated virus vectors, lentivirus vectors, retrovirus vectors and herpesvirus vectors.

The transgene referenced in methods, constructs, and animals described herein can comprise (or encode) any molecule whose expression is desired in a specific population or sub-population of cells. In one embodiment, the transgene is (or encodes) a therapeutic molecule, a toxin or a maker protein. The therapeutic molecules can be any of a number of types of molecules, including, but not limited to a wild-type gene or a pro-apoptotic molecule. In another embodiment, the transgene comprises the viral genes necessary for packaging of an infectious virus.

In one embodiment, the recombinase is expressed in a first population of cells and the transcription factor is expressed in a second population of cells, wherein the first and second populations of cells comprise at least one overlapping sub-population (or subset) of cells and at least one non-overlapping sub-population (or subset) of cells. In one example, the transgene is expressed only in the at least one overlapping sub-population of cells. In another example, the transgene is expressed only in the at least one non-overlapping sub-population of cells.

In one embodiment, the transcription factor is disabled by expression of the recombinase. In one example, the second heterologous nucleic acid comprises recombinase recognition sites flanking the transcription factor, such that expression of the transcription factor is disabled by excision of the transcription factor by the recombinase.

In another embodiment, expression of the transcription factor is enabled by expression of the recombinase. In one example, the second heterologous nucleic acid comprises a transcriptional STOP signal preceding the transcription factor that prevents expression of the transcription factor, wherein the transcriptional STOP signal is flanked by recombinase recognition sites, such that expression of the transcription factor is enabled by excision of the transcriptional STOP signal by the recombinase.

The recombinase can be any recombinase known in the art. For example, the recombinase can be a Cre recombinase, a flp recombinase or a β-recombinase. In addition, the transcription factor can be any transcription factor known in the art. In one embodiment of the methods, the transcription factor is a tTA transcription factor or a fusion protein thereof. When tTA is the transcription factor, the response element can be a tetO transcription response element. In one embodiment, the transcription factor binds to the response element in the absence of an inducing agent. In another embodiment, the transcription factor binds to the response element in the presence of an inducing agent. The appropriate inducing agent is selected based upon which transcription factor and response element are used. For example, if the transcription factor and response element are tTA and tetO, respectively, the inducing agent is selected from tetracycline, doxycycline, or a bioactive derivative thereof.

In one embodiment, at least one of the first and second selective promoters comprises a tissue specific promoter. In another embodiment, at least one of the first and second selective promoters comprises a temporally specific promoter. In another embodiment, at least one of the first and second selective promoters comprises an inducible promoter. In one embodiment, at least one of the first and second selective promoters comprises a promoter that is selective for neural cells. In another embodiment, at least one of the first and the second selective promoters are selective for non-neural cells. In another embodiment, at least one of the first and the second selective promoters are selective for cancer cells. In one aspect, the cancer cell is an AML stem cell. In another embodiment, at least one of the first and the second selective promoters are selective for a population of immune cells. In one aspect, the immune cells are lymphocytes. In another embodiment, at least one of the first and the second selective promoters are selective for infected cells. In another embodiment, at least one of the first and the second selective promoters are selective for a cell comprising a genetic mutation.

Also provided herein are kits for use with subtractive transgenics. In one embodiment, the kit comprises (1) a first viral vector comprising a nucleic acid encoding a recombinase operably linked to a nucleic acid encoding a first selective promoter; (2) a second viral vector comprising a nucleic acid encoding a transcription factor operably linked to a nucleic acid encoding a second selective promoter; and (3) a third viral vector comprising a transgene under the transcriptional control of a response element specific for the transcription factor.

Further provided are kits comprising nucleic acids for the generation of subtractive transgenic animals. Such kits can include injection constructs for the production of a transgenic animal. In one embodiment, the kit comprises (1) a first injection construct comprising a nucleic acid encoding a recombinase operably linked to a first selective promoter; (2) a second injection construct comprising a nucleic acid encoding a transcription factor operably linked to a second selective promoter, wherein expression of the transcription factor is regulated by expression of the recombinase; and (3) a third injection construct comprising a transgene under the transcriptional control of a response element specific for the transcription factor. In one embodiment, the second injection construct comprises the nucleotide sequence of SEQ ID NO: 22. In another embodiment, the second injection construct comprises the nucleotide sequence of SEQ ID NO: 23. In another embodiment, the second injection construct comprises the nucleotide sequence of SEQ ID NO: 24. In another embodiment, the second injection construct comprises the nucleotide sequence of SEQ ID NO: 25. In another embodiment, the second injection construct comprises the nucleotide sequence of SEQ ID NO: 26. In another embodiment, the second injection construct comprises the nucleotide sequence of SEQ ID NO: 27.

### IV. Subtractive Transgenic Animals

The present disclosure further concerns a strategy for producing non-human animals capable of expressing a transgene with a specificity (e.g., an anatomical, spatial, cell, tissue and/or temporal specificity) exceeding that achieved using native promoters. The approach described herein is designated “subtractive transgenics,” because the expression specificity of one promoter is subtracted from that of another. In the subtractive transgenic animals, a transgene of interest is expressed with consistently higher specificity as compared to native promoters. Subtractive transgenics combines two technologies, a site specific recombinase system (such as the Cre/lox system) and an inducible promoter system (such as the tTA/tetO system). In combination, these two systems can be used to increase the specificity, such as the anatomical specificity, of transgene expression. Individually, these systems involve crossing a “trans” line (in which the functional effect depends upon protein expression) to a “cis” line (in which the functional element is a DNA sequence). In both systems, the resultant specificity depends upon the promoter expressing the trans element.

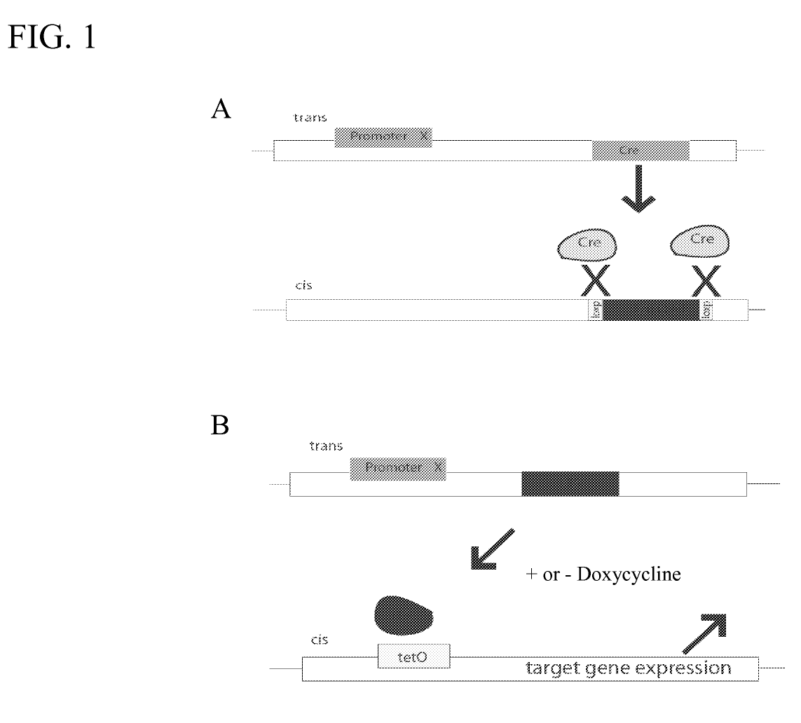

In a site-specific recombinase system, such as the Cre/lox system (for a review of this system see Nagy, Genesis 26(2):99-109, 2000), the trans element is the recombinase (Cre), which can excise any sequence in the genome that is flanked by the corresponding recombinase recognition (loxp) sites (FIG. 1A). This approach has previously been used to make anatomically-specific knockouts, for example, by crossing a mouse expressing a recombinase under the control of a neuron-specific promoter to one in which recombinase recognition sites have been introduced into on either side of an endogenous target gene via homologous recombination in embryonic stem (ES) cells (see, for example, Nakazawa et al. Science 297(5579):211-8, 2002). In the resulting crossed transgenic animals, the target gene is excised only where and when the recombinase is expressed. Suitable site-specific recombinase systems include the Cre/lox system disclosed in, for example, U.S. Pat. No. 6,570,061; the FLP/frt system disclosed in, for example, U.S. Pat. Nos. 6,774,279 and 5,654,182; and the β-recombinase system disclosed in, for example, U.S. Pat. No. 6,780,644. Each of these patents is incorporated herein by reference in its entirety.

An exemplary inducible promoter system is the tTA/tetO system (FIG. 1B), although any regulatory system with analogous regulatory elements is equally suitable in the context of this disclosure. In the tTA/tetO system a transgenic animal (such as a mouse) expressing the trans element (such as the tTA fusion protein) under the control of an exogenous promoter is crossed to a transgenic animal in which a transgene is placed under the regulatory control of the tetO element. When tTA binds to the tetO element, the transgene is expressed. The binding of tTA is tetracycline-dependent, enabling the inducible expression of the transgene wherever the tTA protein is expressed. With tTA, the addition of tetracycline (or its analog doxycycline) turns transgene expression off. The same principles apply in the closely-related rtTA system, but the effect of tetracycline is reversed (Gossen and Bujard, Annu. Rev. Genet. 36:153-73, 2002; and U.S. Pat. Nos. 5,589,362; 5,789,156; 5,654,168; 5,866,755; 5,859,310; and 5,922,927; each of which is incorporated herein by reference).

Transgenic animals have been produced with these systems utilizing neuron-specific promoters (Schonig et al., Nucl Acids Res. 30, No. 23, e134, 2002; Yu et al., Proc. Natl. Acad. Sci. USA 102:8615-8620, 2005). For example, Cre recombinase has previously been expressed under the control of the tetO element, creating inducible knockouts. This is a triple cross in which one line expresses tTA under the control of a native promoter, but the transgene under the control of the tetO element is Cre recombinase. When crossed to another line containing an endogenous gene flanked by loxP recognition sites, recombinase-mediated excision of the endogenous gene is as anatomically specific as the native promoter. In addition, Cre expression is tetracycline-dependent, ensuring that recombination only occurs at the time selected by the practitioner. However, in all such previously-reported cases the anatomical restriction of transgene expression is dependent upon a single promoter construct, and so can therefore only be as specific as the native promoter itself.

“Subtractive transgenics” as provided herein enables the production of transgenic animals with predictable, highly specific patterns of inducible gene expression in distinct cellular subpopulations (or subsets) by subtracting the expression pattern of one promoter from that of another. Accordingly, one aspect of this disclosure concerns methods for producing non-human transgenic animals that express a transgene in a selected subset of cells. The method involves identifying at least one progeny of a cross between a first transgenic non-human animal that contains in its genome a first heterologous nucleic acid (transgene) that encodes a recombinase operably linked to a first selective promoter and a second transgenic non-human animal that contains in its genome a second heterologous nucleic acid (transgene) that encodes a transcription factor operably linked to a second selective promoter. The polynucleotide sequence that encodes the transcription factor is either flanked by site-specific recombinase recognition sites or preceded by a transcription STOP signal that is flanked by site-specific recombinase recognition sites. The identified progeny contains in its genome both of these heterologous nucleic acids, and further a transgene that is under the regulatory control of a response element specific for the transcription factor. Such a transgenic animal expresses the transgene in a highly specific manner only in those cells in which the transcription factor is expressed, and in a manner that is dependent on expression of the recombinase. Based on the selection of promoters, expression can be in any specified sub-population of cells. The animals can be any non-human animals, including mammals and other vertebrates. For example, the transgenic animals can be rodents, such as mice; domesticated livestock, such as cows, sheep, goats, pigs or horses; companion animals; endangered species; and so forth. Similarly, the non-human animal can be a non-human primate. Transgenic animals of any species that possess in their genomes (a) a heterologous nucleic acid that encodes a recombinase operably linked to a first selective promoter; (b) a heterologous nucleic acid that encodes a transcription factor operably linked to a second selective promoter, expression of which is dependent on expression of the recombinase; and (c) a transgene under the transcriptional control of a response element specific for the transcription factor are also features of this disclosure, as are cells derived from such animals.

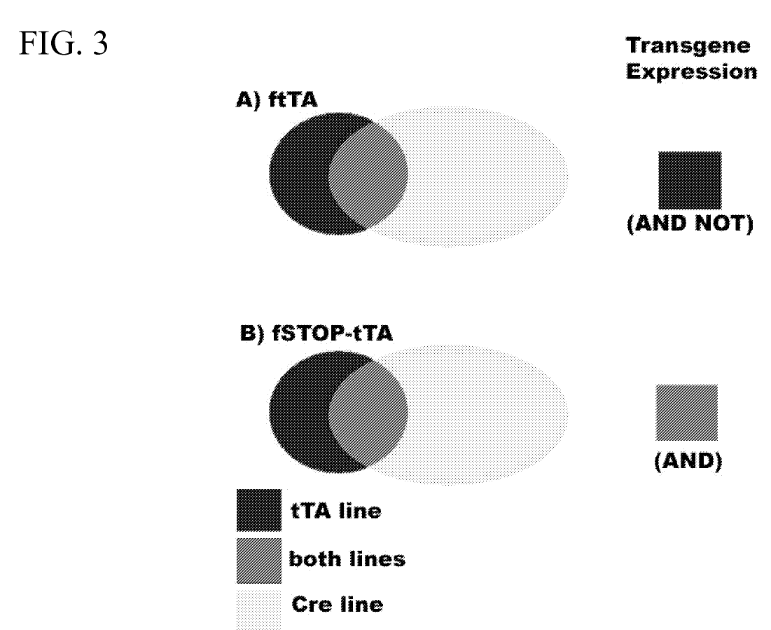

In exemplary embodiments, transgenic lines are established that incorporate a transcriptional activator (such as tTA) that has been flanked by recognition sites for a site specific recombinase (such as Cre), or that has been preceded with a transcriptional STOP signal flanked by such recognition sites, under the control of one selective (for example, spatially or temporally selective) promoter. The resulting line (the “ftTA” or “fSTOP-tTA” line, as exemplified herein) is crossed to a line expressing the corresponding recombinase (e.g., Cre recombinase) with a partially overlapping expression pattern. In the case of an ftTA line, wherever Cre is expressed, the tTA construct will be disabled (excised), resulting in expression of tTA (and thereby the ability for tetracycline-dependent transgene expression) only where transgene expression in the two lines does not overlap. The converse is true in the case of an fSTOP-tTA line, which expresses tTA only where expression of the Cre and tTA lines overlap due to excision of the transcriptional STOP, enabling transcription of tTA. Thus, the ftTA strategy generates an expression pattern analogous to an AND NOT Boolean logic, in which a transgene under control of the tetO element is inducibly expressed only in cells that express tTA but not Cre (FIG. 3A), while the fSTOP-tTA strategy operates by a Boolean AND logic, enabling transgene expression only where the fSTOP-tTA and Cre lines are both expressed (FIG. 3B).

Essentially any site-specific recombinase can be utilized in the methods disclosed herein. For example, non-limiting examples of site-specific recombinases include the Cre recombinase, the FLP recombinase and β-recombinases. Similarly, essentially any transcriptional factor can be used. In specific embodiments, the transcription factor is a transcription activator that binds to its response element in the presence of an inducing agent (for example, the tetracycline dependent tTA transcription factor binds to the tetO response element in the presence but not in the absence of tetracycline, or a bioactive derivative thereof, such as doxycycline). In addition, any promoter or pair of promoters can be utilized in the methods disclosed herein so long as the promoter(s) generate at least a partially overlapping expression pattern. Typically, the promoter(s) are selective promoters that direct expression in a spatially selective or temporally selective manner. For example, a spatially selective expression pattern can include expression in a specified tissue, a specified organ or one or more specified populations of cells. Thus, the present disclosure describes a method in which the specificity of expression of an inducible transactivator (for example, tTA), and therefore the regulated expression of any transgene, is substantially enhanced relative to native promoters.

In brief, an exemplary method described herein involves three distinct steps, although it is noted that the crossing steps can be performed in any order:
1) Generation of animals with “floxed” tTA (ftTA) and/or floxed STOP-tTA (fSTOP-tTA) under the control of a specific promoter and the generation of animals with Cre recombinase under the control of a different specific promoter;
2) Crossing the ftTA and/or fSTOP-tTA lines to animals expressing the Cre recombinase with partially-overlapping expression patterns; and
3) Crossing the resulting “subtracted” lines to a line expressing a transgene under the control of the tetO promoter.

The basic strategy of one exemplary embodiment is to “flox”, or precede with a floxed transcriptional STOP sequence, the tTA injection construct, and then cross the resulting “ftTA” or “fSTOP-tTA” line to another line that expresses the Cre recombinase with a partially overlapping anatomical distribution. Wherever Cre is expressed, tTA is excised or activated, respectively. In this way, the expression pattern of one promoter construct is “subtracted” from that of another. The tTA protein, and therefore the ability to express a transgene from the tetO promoter, remains only in those sub-populations of cells where the two lines do (in the case of fSTOP-tTA) or do not (in the case of ftTA) overlap. The resulting subtracted animal lines thereby exceed the specificity of either parent line—in that the expression of the transgene is more narrowly, or more specifically, regulated. By selecting appropriate promoters (and/or pairs of promoters), the methods provided herein make it possible to manipulate individual cell types, such as single elements of neural circuits.

Animals carrying transgenes with such selectivity of expression are useful for a wide variety of purposes, including the development of model systems for human disorders, such as neurological disorders including Alzheimer's disease, Parkinson's disease and Huntington's disease that affect a specific subset of neural cells with devastating effect. For example, a “silencer” construct (for example, a transgene capable of reducing cell response to stimuli) can be expressed with enhanced specificity, providing a powerful tool for the analysis and influencing of functional relationships between cells, such as in central neural circuits, and making it possible to inactivate part of a functional unit (for example, while recording the ramifications of this inactivation).

In one embodiment, the methods described herein generate lines of transgenic animals capable of temporally and spatially regulated expression of transgene(s). The methods consistently and reproducibly yield increased specificity of transgene expression, such as is sufficient for the analysis of neural circuits. Although the examples and discussion provided throughout this disclosure focus on the expression of transgenes in selected neural cells, the disclosed methods are equally applicable to producing transgenic animals that express a transgene in selected non-neural cells.

Depending upon the specific promoter (or promoter pairs) chosen, the disclosed methods result in increased temporal and/or cell or tissue-type specificity of transgene expression. This subtractive system is modular and combinatorial; by choosing different promoter pairs for the Cre and tTA lines, one can direct expression of any tetO-driven transgene to a virtually unlimited number of cell subtypes. The method does not require that the constituent promoters express in a limited number of cell types, just that the constituent promoters express in some, but not all, the same cell types resulting in an area (that is, a subset of cells) of no overlap that is more limited than full expression scope of the selected promoter(s). While not every promoter pair will result in restriction to a single cell type, every promoter pair will have greater specificity than the parent promoters. Additionally, the subtractive system can also incorporate new advances in promoter specificity (for example, based upon identification of neuron-specific enhancers) as they arise.

Furthermore, as an alternative to (or additive to) employing pairs of promoters with overlapping specificities, a practitioner can take advantage of variability in expression patterns observed in lines of transgenic animals developed from independent founders that exhibit insertional variability in their expression patterns. Typically, insertional variability results in similar, but often non-identical expression patterns in founder animals (and their progeny) with different transgene integration sites. A practitioner of ordinary skill in the art can readily produce multiple lines of transgenic animals using the same transgene construct, characterize the expression pattern on a cellular level, and select appropriate lines that provide or will result in (e.g., after one or more additional crosses) the desired overlapping expression specificity.

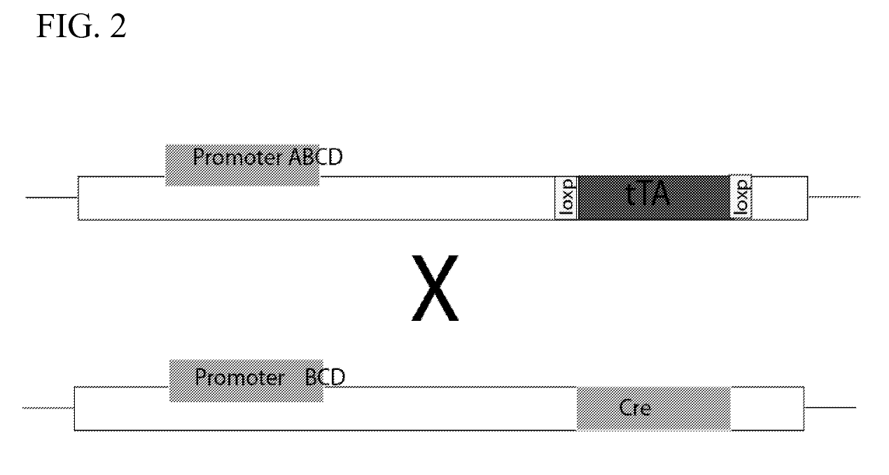

As illustrated schematically in FIG. 2, a transgenic animal that has a floxed tTA (ftTA) under the control of a promoter that confers expression in cells A, B, C, D is crossed with another transgenic animal that has the Cre recombinase regulated by a promoter that expresses in the same cells B, C, D (and can also express in non-overlapping cells E, F, G etc.) but does not express in cells A. Crossing the two transgenic animals produces progeny in which tTA expression occurs only in cell type A, which can then specifically drive expression of a transgene under the transcriptional control of the tetO response element.

In contrast, when an fSTOP-tTA animal under the control of a promoter that expresses in cells A, B, C and D is crossed with a transgenic animal that expresses Cre in cells B, C and D, the animal expresses tTA in cells B, C and D and is capable of inducibly expressing a transgene under tetO regulation in these same cells.

In certain examples, the transgene is a “silencer” (see, for example, Ibanez-Tallon et al. Neuron 43(3):305-11, 2004). For example, by expressing a silencer under the control of the tetO response element in specific cells of the nervous system, it is possible to reversibly remove a neuron from participating in a circuit, either by preventing it from spiking or by interfering with neurotransmission, in a manner analogous to shorting out an electronic circuit element. This molecular short-circuit provides a relatively unambiguous and demonstrable effect on both the cellular and network level. Once a line is confirmed to possess the desired expression pattern, the extent to which neuronal silencing occurs is determined by a combination of extracellular unit or field recordings in vivo and field and intracellular recordings in vitro. In addition to yielding a wealth of electrophysiological data regarding the functional attributes of specific neural circuits, the methods and animals disclosed herein are also useful for generating more accurate and informative models of clinical disorders.

While any transgene can be expressed with maximal specificity using the methods disclosed herein, these methods are particularly useful for expressing genes (including modified genes) that model human disease within or outside the nervous system. For example, overexpression of proteins, such as amyloid precursor protein (APP) or presenilins (for example, Arancio et al. EMBO J. 23(20):4096-4105, 2004) relevant to Alzheimer's disease can be achieved with sufficient temporal and anatomical specificity to evaluate their primary and secondary effects. Similarly, molecules that have neuroprotective or anti-aging effects can be expressed with similar control, allowing comparison of protective effect relative to ischemic insult or aging between adjacent neurons. Additionally, transgenic animals produced according to the methods disclosed herein are useful for evaluating specific roles played by cells that have been compromised by trauma in order to develop strategies (such as implanting prostheses) to mitigate cellular loss.

A. Promoter Selection

In the production of transgenic animals or cell lines, it is frequently observed that different lines established from founders with independent transgene insertion events have similar (for example, overlapping) but non-identical transgene expression patterns. Thus in the context of subtractive transgenics, the same promoter can be used to produce multiple lines, which are then characterized with respect to transgene expression pattern. Two lines (a transcription factor line and a recombinase line) with non-identical expression patterns that differ with respect to expression in a cell type(s) of interest are selected and crossed to produce progeny that are capable of expressing the transgene exclusively in the cell type(s) of interest. This selection process is well within the ability of one of ordinary skill.

Thus, promoter selection can be influenced by taking advantage of the insertional variability in expression patterns that result from multiple founders made using the same promoter. Since these lines often express in slightly different subsets of cells than the native promoter, insertional effects become a valuable source of expression specificity diversity for the subtractive transgenic method. For example, as discussed further in the section entitled “EXAMPLES,” the Calcium-Calmodulin-dependent Protein Kinase II alpha subunit (CamKIIα) promoter expresses strongly in a variety of neuronal cell types in the forebrain, and has yielded significant variability in expression patterns that arise in different founders made using the same construct. Although simply selecting a line with a desirable anatomical (and/or temporal) expression pattern has been useful to increase specificity of expression, the expression patterns obtained typically involve more than a single neuron type. With a subtractive strategy as provided herein, however, this variability can be used combinatorially to produce a host of different cell-restricted expression patterns in different areas, including areas of the brain that are particularly interesting from a cognitive and clinical viewpoint (for example, cortex, striatum, hippocampus, and so forth).

Another method for selecting suitable promoter(s) involves utilizing the National Institutes of Health (NIH)-funded resource called Gene Expression Nervous System Atlas, or GENSAT project (on the world wide web at GENSAT.org, supported by NINDS Contract #NO1-NS-0-2331), located at Rockefeller University. The GENSAT project has created lines of transgenic mice that express an enhanced green fluorescent protein (GFP) reporter gene from hundreds of different promoters using bacterial artificial chromosomes (BACs), and anatomically characterized the resulting GFP expression pattern. The resulting lines not only demonstrate the expression pattern governed by the promoter, but also show whether a particular promoter is capable of reproducibly driving a transgene with a consistent (or variable) expression pattern.

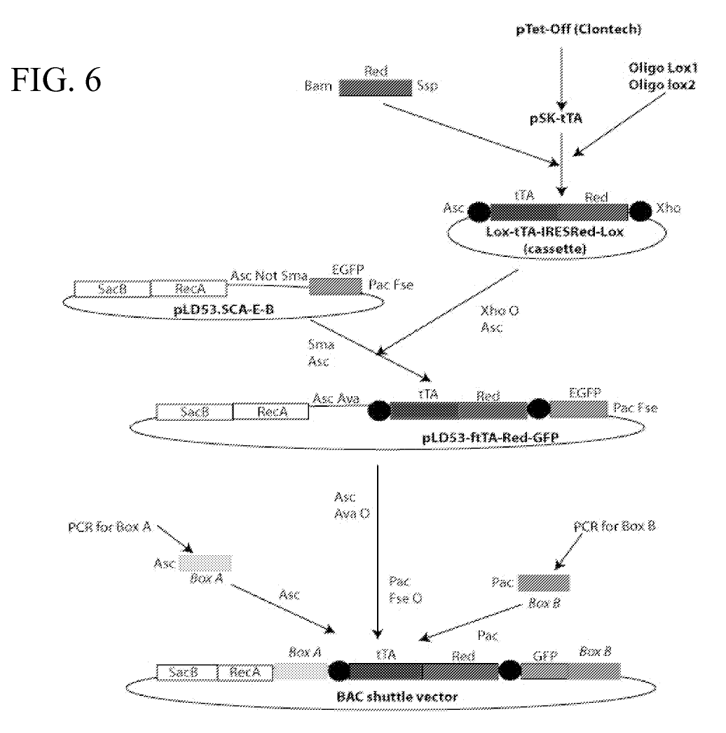

For example, once a suitable promoter has been selected from among those catalogued in the GENSAT project, an ftTA or fSTOP-tTA construct can be produced in which the floxed tTA (or floxed STOP tTA) encoding sequences are used (such as in place of GFP) in an analogous shuttle vector. In brief, a BAC construct is made by selecting a BAC clone 5′ to the gene of interest and then preparing shuttle vectors specific to that BAC clone (FIG. 6). The shuttle vectors can be RecA positive selectable targeting constructs (such as those based upon the Pld53 backbone, see Gong et al. Nature 425(6961):917-925, 2003) in which the DNA to be inserted is flanked by one or two stretches of sequence identical to contiguous sequences in the BAC (the A and B box, see FIG. 6). The targeting construct is then electroporated into host bacteria containing the BAC clone of interest. Integration of the targeting vector into the BAC by homologous recombination is then assayed by exposure to the appropriate selection agent; vector sequences can be resolved by a second round of selection. A floxed tTA (or fSTOP-tTA) construct can be produced by ligating the floxed tTA (or fSTOP-tTA sequences) into the shuttle vector. Optionally, the GFP of the GENSAT vector can be retained.

These lines express ftTA with the same specificity as the native promoter. Furthermore, if the GFP is retained, the “subtracted” neurons express GFP, facilitating analysis of anatomical and/or temporal restriction of expression.

Optionally, additional markers can be added to the construct to facilitate analysis of the subtracted lines. For example, the Piresii-red2 reporter (Clontech, Mt. View, Calif.) can be inserted into the vector in place of or in addition to GFP (FIG. 6). Alternatively, the mOrange fluorophore (Shaner et al., Nature Biotechnol 22(12) 1554 2004) can be added to the ftTA construct either as a fusion protein or by putting an internal ribosome initiating site (IRES) 3′ of the tTA sequence but 5′ of the mOrange sequence, which would also be in turn 5′ of the 3′ loxp site. Inclusion of such additional fluorophores results in a subtracted line with red (or orange) cells that express the tTA transgene, in contrast to the green ones that have been subtracted, obviating the need for in situ hybridization to characterize the transgenic lines.

Suitable promoters and/or pairs of promoters can also be identified by reviewing the literature and selecting promoters having the desired expression characteristics. However, in many cases, the published expression patterns lack a high degree of resolution. For many transgenes, the only expression data comes from in situ hybridization, often visualized simply by allowing S³⁵-labelled tissue slices to expose X-ray film placed on top of the slide. While radiographic methods have the benefit of being relatively quantitative, it is difficult to determine anything finer than what can be determined from gross anatomical structures that show signal with X-ray film. Excellent anatomical information can also be obtained from emulsion-dipped slides of radioactive in situ hybridizations, which combines the quantitative accuracy of radiography with cellular resolution. In any case, a practitioner may desire to further characterize the expression profile of a selected (or potential) promoter in order to ensure that the characteristics are as desired for the subtractive expression procedure being used or developed.

Alternatively, promoters can be selected by characterizing de-novo the expression pattern in one or more lines of transgenic animals that express a transgene under the control of the promoter in question.

B. Crossing of Transgenic Animals

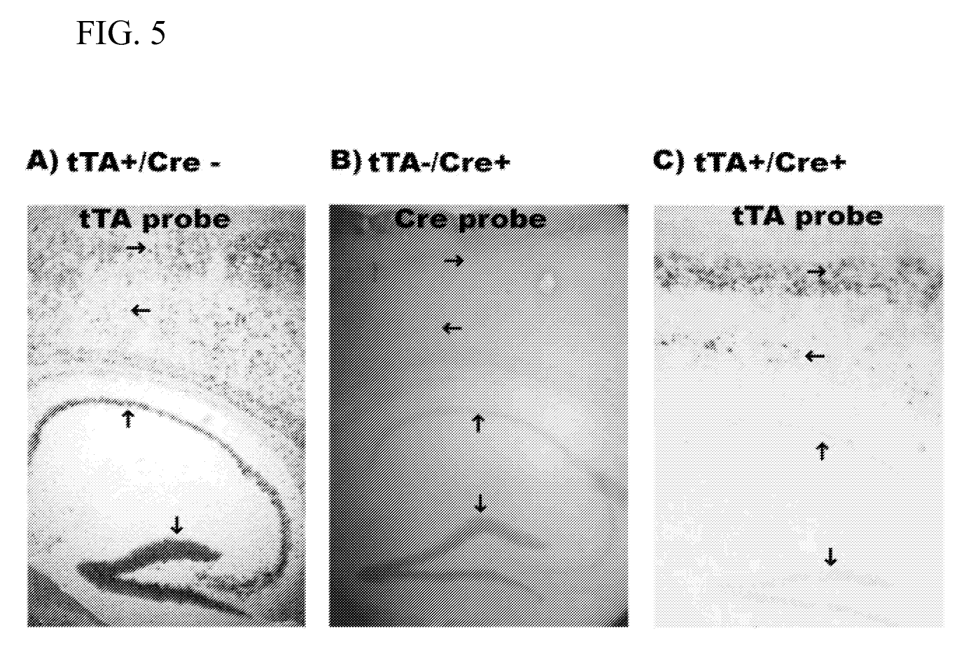

In one exemplary embodiment, subtraction of expression patterns requires crossing a floxed tTA line to a complementary Cre line, for example, a line in which expression of Cre overlaps with tTA expression in all but one neuronal cell type. FIG. 5 illustrates gene expression in two lines of mice with overlapping expression of particular interest for generating hippocampal-specific transgene expression via subtraction. For example, mating an ftTA line that expresses only in dentate and CA1 to the Cre line shown in FIG. 5C results in dentate-specific expression, whereas mating an ftTA line that expresses in dentate, CA1, and CA3 results in CA3-specific expression of the transcriptional activator.

Following identification of progeny that express the transcription factor in the desired cells, the identified progeny are crossed with a transgenic line that expresses a transgene under the control of a response element specific for the transcription factor. In the exemplary embodiment utilizing the tTA transcription factor, the transgene is placed under the regulatory control of the tetO response element. Placing a transgene under the control of the tetO response element enables expression of the transgene in a regulated fashion based upon the presence or absence of doxycycline in the animal's diet. Essentially any transgene of interest can be used in this context. For example, one class of transgene includes silencer constructs that “turn off,” or inhibit the activity of cells, such as toxins, dominant negative constructs, and inhibitory RNA molecules. In other examples, the transgenes are particular nucleic acids of interest with respect a particular cell type, condition, and/or disease. In yet other examples, the transgenes are markers (such as reporters).

The transgene is inserted into a convenient cloning site (such as NotI) of a tetO vector (Pmm400) or into an equivalent restriction site preceded by any response element inducible by a transcription factor. The resulting animals are mated to an animal of a subtracted line (generated by crossing Cre and tTA lines with overlapping expression patterns) described herein. The resulting transgenic progeny express the transgene in a tetracycline-dependent manner in the specified neural cells.

In certain examples, the transgene is a neural silencer. There are several different types of expressible silencer constructs available, ranging from inward-rectifier K+ channels, which basically decrease the excitability of the neuron, to those which specifically interfere with synaptic transmission. For example, one type of a silencer suitable for use in the context of the subtractive transgenic animals described herein is the nontoxic heavy chain of tetanus toxin described by Maskos et al. (Proc. Natl. Acad. Sci. U.S.A. 99(15):10120-10125, 2002). The heavy chain of tetanus toxin is involved in transynaptic transport. The particular derivative described by Maskos has been fused to GFP, thereby creating an expressible retrograde transynaptic marker that is effectively expressed in transgenic animals when operably linked to the calbindin minimal promoter. Additionally, the tetanus toxin-GFP fusion protein also includes lacZ via an IRES site, in effect creating a construct capable of differentiating between the initial cell (which would be GFP+ and lacZ+) and its upstream inputs (which would express GFP only).

Additional silencers include dominant negative transgenes which are capable of functional removal of the cells in which they are expressed. In neural cells, there are two basic types of functional silencer, each of which can be either ligand-gated or constitutive: (1) silencers of spiking, and (2) silencers of neurotransmission. Some silencers inhibit spiking by decreasing the input resistance of the membrane and thereby shunting excitatory current, or by providing a hyperpolarizing current which counteracts synaptic depolarization, preventing the membrane from reaching spike threshold. Examples of ligand-gated silencers include the ivermectin-sensitive chloride channel developed by Slimko and Lester (FEBS Lett. 528(1-3):77-82, 2002), the insect allatostatin receptor (Lechner et al., J. Neurosci. 22(13):5287-5290, 2002) and VAMP-MIST constructs (Karpova et al., Soc. Neurosci Program No. 808.1, 2004; Karpova et al. Neuron 48(5):727-735, 2006), composed of a synaptic vesicle protein susceptible to a chemical crosslinking agent (the “ligand”). Silencers which act at the level of neurotransmission, such as the shibire(ts) allele of Drosophila (see, Kitamoto, Proc. Natl. Acad. Sci. U.S.A. 99(20):13232-13237, 2002), prevent neither depolarization nor associated calcium entry, rather they work by inhibiting the release of synaptic vesicles. The tetanus toxin light chain is more specific as it proteolytically destroys a protein needed for vesicle release (VAMP2, Yu et al. Neuron 42(4):553-66, 2004).

Other examples of silencers include a “tethered” ω-conotoxin MVIIA or μ-conotoxin MrVIA. These constructs have been demonstrated to specifically block voltage-gated Ca′1 and Na+ channels only in the cells in which they are expressed (Ibanez-Tallon et al. Neuron 43(3):305-11, 2004).

C. Generating Transgenic Animals

Any transgenic animal can be of use in the methods disclosed herein, provided the transgenic animal is a non-human animal. A “non-human animal” includes, but is not limited to, a non-human primate, a farm animal such as swine, cattle, and poultry, a sport animal or pet such as dogs, cats, horses, hamsters, rodents, or a zoo animal such as lions, tigers or bears. In one specific, non-limiting example, the transgenic animal is a mouse.

A transgenic animal contains cells that bear genetic information received, directly or indirectly, by deliberate genetic manipulation at the subcellular level, such as by microinjection or infection with a recombinant virus, such that a recombinant DNA is included in the cells of the animal. This molecule can be integrated within the animal's chromosomes, or can be included as extrachromosomally replicating DNA sequences, such as might be engineered into artificial chromosomes. A transgenic animal can be a “germ cell line” transgenic animal, such that the genetic information has been taken up and incorporated into a germ line cell, therefore conferring the ability to transfer the information to offspring. If such offspring in fact possess some or all of that information, then they, too, are transgenic animals.

Transgenic animals can readily be produced by one of skill in the art. For example, transgenic animals can be produced by introducing into single cell embryos DNA encoding a marker, in a manner such that the polynucleotides are stably integrated into the DNA of germ line cells of the mature animal and inherited in normal Mendelian fashion. Advances in technologies for embryo micromanipulation permit introduction of heterologous DNA into fertilized mammalian ova. For instance, totipotent or pluripotent stem cells can be transformed by microinjection, calcium phosphate mediated precipitation, liposome fusion, retroviral infection or other means. The transformed cells are then introduced into the embryo, and the embryo then develops into a transgenic animal. Developing embryos can also be infected with a retrovirus containing the desired DNA, and a transgenic animal is produced from the infected embryo.

In another example, the appropriate DNA(s) can be injected into the pronucleus or cytoplasm of embryos, preferably at the single cell stage, and the embryos are allowed to develop into mature transgenic animals. These techniques are well known. For instance, reviews of standard laboratory procedures for microinjection of heterologous DNAs into mammalian (mouse, pig, rabbit, sheep, goat, cow) fertilized ova include: Hogan et al., Manipulating the Mouse Embryo, Cold Spring Harbor Press, 1986; Krimpenfort et al., Bio/Technology 9:86, 1991; Palmiter et al., Cell 41:343, 1985; Kraemer et al., Genetic Manipulation of the Early Mammalian Embryo, Cold Spring Harbor Laboratory Press, 1985; Hammer et al., Nature 315:680, 1985; Purcel et al., Science 244:1281, 1986; U.S. Pat. No. 5,175,385; U.S. Pat. No. 5,175,384, each of which is herein incorporated by reference.

### V. Subtractive Transgenics in Animals Using Viral Vectors

Subtractive transgenics can also be applied to whole animals through the use of suitable viral vectors. To use subtractive transgenics in animals (as opposed to generating transgenic animals by introducing constructs into an ES cell), the system components are delivered using viral vectors. As with the production of transgenic animals described herein, three different viral vector constructs are employed: (1) a transactivator virus; (2) a recombinase virus; and (3) a transgene virus. The transactivator virus encodes a transactivator, such as tTA or a transcription factor. In addition, the transactivator virus comprises recombinase recognition sites. The recombinase recognition sites can either flank the transactivator, or they can flank a STOP signal that precedes the transactivator, much as described elsewhere herein. The recombinase virus encodes a recombinase (that recognizes the recombinase recognition sites in the transactivator virus). Any suitable recombinase can be used, such as Cre, FLP or β-recombinase. The transgene virus encodes any type of transgene of interest, wherein the transgene is regulated by an appropriate response element, much as described elsewhere herein. The response element employed depends upon the transactivator used for the transactivator virus. For example, if the transactivator virus encodes tTA, then the response element would be tetO.

The transactivator virus and recombinase virus can each be regulated by different specific promoters (such as tissue-specific, cell-specific or temporal-specific promoters). As described herein, the use of promoters exhibiting different expression patterns allows for the subtraction of the population of cells expressing one construct from the population of cells expressing the other construct.

A. Viral Vectors for Use with Subtractive Transgenics

Suitable viral vectors include, but are not limited to, adenovirus vectors, adeno-associated virus vectors, retroviral vectors, lentiviral vectors, herpesviral vectors, and the like.

Adenovirus vectors can be first, second, third and/or fourth generation adenoviral vectors or gutless adenoviral vectors. Adenovirus vectors can be generated to very high titers of infectious particles; infect a great variety of cells; efficiently transfer genes to cells that are not dividing; and are seldom integrated in the host genome, which avoids the risk of cellular transformation by insertional mutagenesis (Douglas and Curiel, Science and Medicine, March/April 1997, pages 44-53; Zern and Kresinam, Hepatology 25(2), 484-491, 1997). Representative adenoviral vectors which can be used for the methods provided herein are described by Stratford-Perricaudet et al. (J. Clin. Invest. 90: 626-630, 1992); Graham and Prevec (In Methods in Molecular Biology: Gene Transfer and Expression Protocols 7: 109-128, 1991); and Barr et al. (Gene Therapy, 2:151-155, 1995), which are herein incorporated by reference.

Adeno-associated virus (AAV) vectors also are suitable for use with subtractive transgenics. Methods of generating AAV vectors, administration of AAV vectors and their use are well known in the art (see, for example, U.S. Pat. No. 6,951,753; U.S. Pre-Grant Publication Nos. 2007-036757, 2006-205079, 2005-163756, 2005-002908; and PCT Publication Nos. WO 2005/116224 and WO 2006/119458, each of which is herein incorporated by reference).

Retrovirus, including lentivirus, vectors can also be used with the methods described herein. Lentiviruses include, but are not limited to, human immunodeficiency virus (such as HIV-1 and HIV-2), feline immunodeficiency virus, equine infectious anemia virus and simian immunodeficiency virus. Other retroviruses include, but are not limited to, human T-lymphotropic virus, simian T-lymphotropic virus, murine leukemia virus, bovine leukemia virus and feline leukemia virus. Methods of generating retrovirus and lentivirus vectors and their uses have been well described in the art (see, for example, U.S. Pat. Nos. 7,211,247; 6,979,568; 7,198,784; 6,783,977; and 4,980,289, each of which is herein incorporated by reference).

Suitable herpesvirus vectors can be derived from any one of a number of different types of herpesviruses, including, but not limited to, herpes simplex virus-1 (HSV-1), HSV-2 and herpesvirus saimiri. Recombinant herpesvirus vectors, their construction and uses are well described in the art (see, for example, U.S. Pat. Nos. 6,951,753; 6,379,6741 6,613,892; 6,692,955; 6,344,445; 6,319,703; and 6,261,552; and U.S. Pre-Grant Publication No. 2003-0083289, each of which is herein incorporated by reference).

B. Administration of Viral Vectors

The viral vectors can be administered to an animal using any suitable means known in the art. Methods of administration include, but are not limited to, intradermal, intramuscular, intraperitoneal, parenteral, intravenous, subcutaneous, vaginal, rectal, intranasal, inhalation, oral or by gene gun. Intranasal administration refers to delivery of the compositions into the nose and nasal passages through one or both of the nares and can comprise delivery by a spraying mechanism or droplet mechanism, or through aerosolization of the nucleic acid or virus. Administration of the compositions by inhalant can be through the nose or mouth via delivery by spraying or droplet mechanisms. Delivery can be directly to any area of the respiratory system via intubation. Parenteral administration is generally achieved by injection. Injectables can be prepared in conventional forms, either as liquid solutions or suspensions, solid forms suitable for solution of suspension in liquid prior to injection, or as emulsions. Injection solutions and suspensions can be prepared from sterile powders, granules, and tablets. Administration can be systemic or local.

Recombinant virus or vector nucleic acids are administered in any suitable manner, preferably with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Administration can be accomplished by single or multiple doses. The dose required will vary from subject to subject depending on the species, age, weight and general condition of the subject, the particular nucleic acid or recombinant virus being used and its mode of administration. An appropriate dose can be determined by one of ordinary skill in the art using only routine experimentation. The three viral vectors can be administered simultaneously or separately. If administered separately, the time between delivery of each virus can vary between seconds, minutes, days, weeks and months between each administration.

Provided herein are pharmaceutical compositions which include one or more viral vectors, alone or in combination with a pharmaceutically acceptable carrier. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate. Any of the common pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used with the compositions and methods provided herein are normal saline and sesame oil.

### VI. Transgenes

Subtractive transgenics allows for predictable, highly-selective expression of a transgene in animals, including humans. Subtractive transgenics can be applied by generating transgenic animals. Alternatively, through the use of viral vectors, subtractive transgenics can be used with whole organisms, without the requirement for generating and crossing transgenic animals. The selected transgene can be any molecule whose expression is desired in a specific population or sub-population of cells. The transgene can be, or encode, any type of molecule, including, but not limited to, a therapeutic molecule, a silencer, an inhibitory RNA, a dominant negative molecule, a toxin, a pro-apoptotic molecule, a wild-type gene, or a marker or reporter gene. The transgene can also encode viral accessory or packaging proteins that enable the production of lytic virus.

A therapeutic molecule is any molecule that prevents, treats or ameliorates a disease, condition or disorder (such as a genetic disorder, infection or cancer). In one embodiment, a therapeutic molecule is a wild-type version of a mutated gene or protein involved in a particular disease or condition. For example, in the case of cystic fibrosis, subtractive transgenics can be used to selectively express a wild-type version of the CFTR gene, which is defective in these patients. As another example, a significant number of cancerous cell types express a mutated form of a tumor suppressor gene, such as p53. Subtractive transgenics can be used to selectively express a wild-type version of the tumor suppressor gene in cancer cells.

In some embodiments, the therapeutic molecule is a silencer. As described herein, silencers include any number of different types of molecules, including toxins, inhibitory RNA, and dominant negative proteins.

A toxin includes any molecule that causes a toxic response (such as cell death) in the population of cells in which it is expressed. Toxins are usually proteins that are capable of causing disease by interacting with biological macromolecules such as enzymes or cellular receptors. Toxins include, but are not limited to botulinum toxin, tetanus toxin and diphtheria toxin.

In another embodiment, the therapeutic molecule is a pro-apoptotic molecule. A pro-apoptotic molecule would serve a therapeutic purpose, for example, for the treatment of a condition in which cell death is the goal, such as cancer or other hyperproliferative disease, or an infection. Using the methods described herein, pro-apoptotic molecules can be specifically delivered to the selected cells to induce cell death. Pro-apoptotic molecules are well known in the art and include, for example, caspases. Caspases are generally divided into two groups, initiator caspases and effector caspases. Initiator caspases, which include caspase-1, caspase-2, caspase-8, caspase-9, caspase-10 and caspase-12, cleave inactive pro-forms of effector caspases, thereby activating. Effector caspases, which include caspase-3, caspase-6, caspase-7 and caspase-14, cleave other protein substrates within the cell resulting in the apoptotic process. Two other known caspases, caspase-4 and caspase-5, are unclassified. Other pro-apoptotic molecules include granzyme B, Fas and Fas ligand, Bcl-2 and Bcl-X (and related pro-apoptotic family members), ceramide, second mitochondria-derived activator of caspases (SMAC) proteins, tumor necrosis factor and death domain containing molecules.

Marker and/or reporter proteins are well known in the art. Such markers or reporters include, but are not limited to green fluorescent protein (or other types of fluorescent marker proteins), chloramphenicol acetyltransferase or luciferase.

### VII. Kits

Also provided herein are kits comprising nucleic acid components for use with subtractive transgenics, which are provided and described herein. In one embodiment, the kit comprises (1) a first viral vector comprising a nucleic acid encoding a recombinase operably linked to a nucleic acid encoding a first selective promoter; (2) a second viral vector comprising a nucleic acid encoding a transcription factor operably linked to a nucleic acid encoding a second selective promoter; and (3) a third viral vector comprising a transgene under the transcriptional control of a response element specific for the transcription factor.

Also provided are kits comprising nucleic acids for the generation of subtractive transgenic animals. Such kits can include injection constructs for the production of a transgenic animal. In one embodiment, the kit comprises (1) a first injection construct comprising a nucleic acid encoding a recombinase operably linked to a first selective promoter; (2) a second injection construct comprising a nucleic acid encoding a transcription factor operably linked to a second selective promoter, wherein expression of the transcription factor is regulated by expression of the recombinase; and (3) a third injection construct comprising a transgene under the transcriptional control of a response element specific for the transcription factor.

In one embodiment, the second injection construct comprises the nucleotide sequence of SEQ ID NO: 22. In another embodiment, the second injection construct comprises the nucleotide sequence of SEQ ID NO: 23. In another embodiment, the second injection construct comprises the nucleotide sequence of SEQ ID NO: 24. In another embodiment, the second injection construct comprises the nucleotide sequence of SEQ ID NO: 25. In another embodiment, the second injection construct comprises the nucleotide sequence of SEQ ID NO: 26. In another embodiment, the second injection construct comprises the nucleotide sequence of SEQ ID NO: 27.

The following examples are provided to illustrate certain particular features and/or embodiments. These examples should not be construed to limit the disclosure to the particular features or embodiments described.

### EXAMPLES

### Example 1

### Granule Cell Specific Expression of a Transgene

In one particular example, a transgene is expressed exclusively in the granule cells of the dentate gyrus. The hippocampal circuit is essentially a loop from entorhinal cortex to dentate gyrus; dentate to CA3; CA3 to CA1; CA1 to subiculum (or subicular complex); and subiculum (and CA1) back to entorhinal cortex (see, for example, Witter et al. Ann. N.Y. Acad. Sci. 911:1-24, 2000). While this describes the majority of synapses in the hippocampal formation, recent evidence has underscored that some of the “minority” connections (particularly entorhinal to CA1) may play a much more important role than previously thought (see, for example, Brun et al. Science 296(5576):2243-6, 2002). The most direct way to see what each major neural circuit element does is to specifically and reversibly ablate it while recording from downstream elements, as one would any other circuit.

Subtractive transgenics can be used to produce a dentate granule cell-specific inducible silencer mouse, in which it is possible to implant electrodes and monitor specific regions (for instance, CA1) while the transgene is off (either with or without doxycycline, for tTA and rtTA, respectively). After stable baseline recordings of CA1 pyramidal cells (for example, “place cells” and “theta” cells) are obtained, doxycycline is added to the diet, thereby initiating the expression of the silencer transgene in dentate granule neurons. In this way, the relative contribution of the firing of granule cells to the firing of CA1 pyramidal cells can be determined. This same line can also be used to analyze the relative contribution of granule cells to other parts of the neural circuit, including CA3 and/or entorhinal cortex, simply by switching the recording site. Furthermore, unit recordings are only one of the many approaches one could take with such animals. Extracellular field recordings before and after transgene induction can also be performed (for example, to elucidate the role of granule cells in theta and gamma-band oscillations), as can current-source-density (CSD) analysis (for example, with multisite recordings such as silicon probes).

In this example, known anatomical variability in lines of mice expressing transgenes under the control of the CamKIIα promoter is utilized in the context of the subtractive transgenic technique to produce transgenic animals that express specifically in the granule cells of the dentate gyrus. Due to the widespread use of the CamKIIα promoter, there are many different lines available that express Cre in overlapping sets of neural cells, including the granule cells of the dentate gyrus.

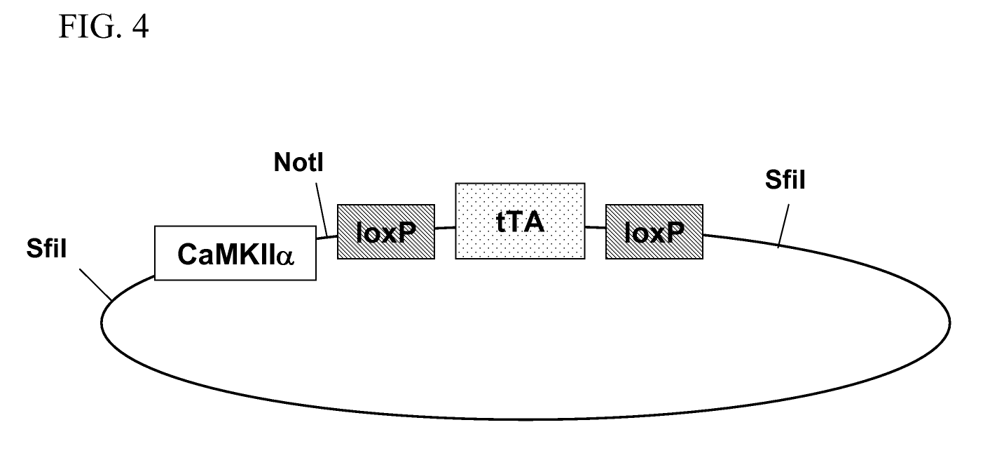

The basic ftTA construct is illustrated in FIG. 4. The ftTA construct was derived from the pMM403 CamKIIα plasmid (Bejar et al. J. Neurosci. 22(13):5719-26, 2002) by flanking it with symmetrical loxp sites in mutagenic PCR reactions with a high-fidelity polymerase. The loxp primers both had Not I linkers, while the internal primers were designed to enable the directional cloning of the floxed ends into the tTA construct. The ftTA construct was then ligated into the Not I site of pMM403. The construct for pronuclear injection was created by cutting this plasmid with SfiI and gel-purifying the relevant (upper) band.

Several lines were established from different transgene positive founder animals using this construct. Non-radioactive in situ hybridization on seven of these lines with ˜600 bp digoxygenin-labeled riboprobes transcribed from the injection construct demonstrated that anatomical variability in expression pattern similar to that previously reported is obtained. Individual lines differ in their expression patterns in neuronal subtypes in the hippocampus, cortex, and striatum.

FIG. 5 shows in situ hybridizations of a subtractive pair of transgenic lines of mice both made with the CaMKII promoter demonstrating proof of principle: increased anatomical specificity via Cre-mediated subtraction. FIG. 5A illustrates the tTA expression (“unsubtracted”) under control of the CaMKII promoter in a transgenic line, with very strong labeling of the principal cells of the entire hippocampus (up and down arrows), and moderately strong labeling in two sets of cortical pyramidal neurons (left and right arrows). FIG. 5B shows the anatomical distribution of Cre in the “subtractor” Cre line, with weak labeling throughout the hippocampus, and no detectable cortical label. FIG. 5C demonstrates the results of crossing these two lines: the strong hippocampal labeling is almost entirely “subtracted.” In the subtracted animals tTA expression is present only in the cortical neurons. Crossing of these subtracted animals to a tetO line produces transgenic animals that are capable of expressing any transgene of interest under the control of the tetO response element only in the cortical neurons.

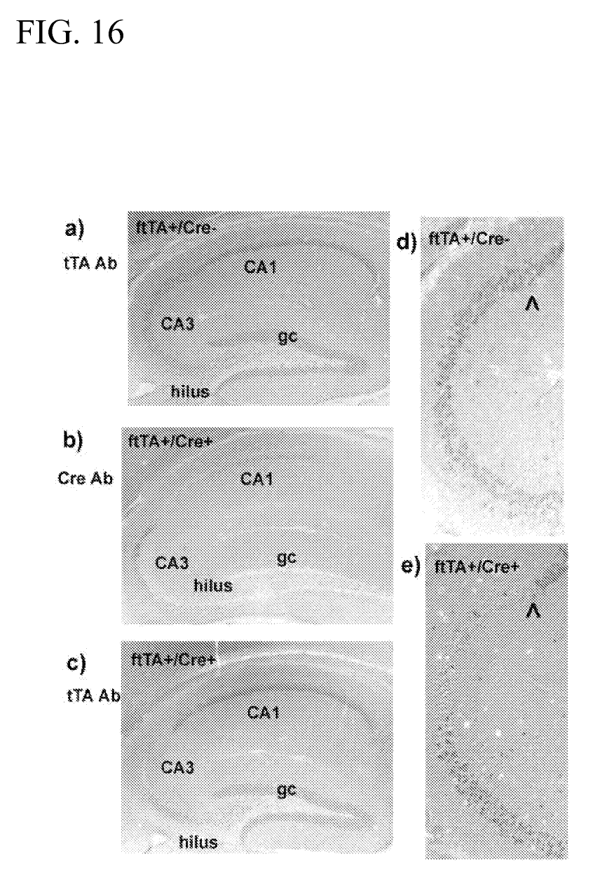

Another example demonstrating the advantage provided by anatomical variability of the CamKIIα promoter is shown in FIG. 16. In this example, one transgenic line comprises Cre under control of the CamKIIα promoter and another line comprises ftTA under control of the CamKIIα promoter. In the unsubtracted line (ftTA⁺/Cre⁻), expression of tTA is detected in all primary neurons (CA1, CA2, gc and hilus). In the subtracted line (ftTA⁺/Cre⁺), Cre expression is detected only in CA3 cells. Thus, subtraction results in expression of tTA in all primary neurons except CA3 (see FIG. 16C).

### Example 2

### Expression in Subsets of Cholinergic Neurons Using Variants of the Choline Acetyltransferase Promoter

In order to demonstrate selectivity using a selected promoter pair, two variants of the choline acetyltransferase (Chat) promoter were selected that have an overlapping expression pattern in a subset of cholinergic neurons. Chat is an enzyme involved in the biosynthesis of acetylcholine, and is expressed only in cholinergic neurons. Cholinergic input arising from the basal forebrain nuclei plays a key role in neuromodulation of the telencephalon, and is thought to be involved with memory, arousal and attention. Loss of central cholinergic tone has been hypothesized to be involved with age-related cognitive decline. Thus, a promoter specific to those particular cholinergic nuclei would be of great general interest. However, the native Chat promoter expresses in every cholinergic neuron in the body, including key brainstem nuclei and spinal motor neurons involved in movement. The native Chat promoter is therefore of limited utility since memory tasks in animal models are frequently involve behavioral tasks with a substantial motor component and it would be difficult to get adequate behavioral data out of an animal with silenced motoneurons.

Because not every 5′ BAC clone recapitulates the native expression pattern (Gong et al. Nature 425(6961):917-25, 2003), it is possible to select particular promoter clones that consistently yield somewhat different expression patterns. For example, two Chat promoter BAC clones yield expression in different (overlapping) cholinergic neurons. BAC RP23-431D19 yields a recapitulation of the native expression pattern, whereas BAC RP23-51F19 yields an incomplete and ectopic expression pattern. In combination, these two BAC promoter clones can be used to produce subtractive transgenic lines that express a transgene in cholinergic neurons of the basal forebrain and striatum, but not in critical motoneurons and brainstem cholinergic neurons.

To produce such subtractive transgenic lines, a Chat ftTA cassette is inserted into BAC RP23-431D19, and a Cre cassette is inserted into BAC RP23-51F19 as illustrated in FIG. 6. The ftTA cassette is created in a shuttle vector (such as a BLUESCRIPT™ vector from Stratagene). First, the tTA fragment EcoRI-BamHI from pTet-Off (Clontech) is inserted in BamHI and EcoRI sites of pBluescript (plasmid SK-tTA). Then, two oligonucleotides incorporating the LoxP sites are added. A first oligonucleotide (Oligo Lox1) is designed to have the following sequence: KpnI-AscI-AvaI-LoxP-ApaI-HindIII. The second oligonucleotide (Oligo LoxP2) has the sequence EcoRV-SpeI-LoxP-XhoI-NotI-EcoRI. The Oligo Lox1 is digested with KpnI and HindIII and inserted in the same sites of the SK-tTA. The Oligo Lox2 is digested by SpeI and NotI and inserted in the same sites of the SK-tTA. Finally, the IRES-Red is extracted from the pIRESII-red2 plasmid by BamHI and SspI and inserted in the SpeI blunted, BamHI sites of the construct. The cassette LoxP-tTA-IRESRed-LoxP is extracted by AscI, XhoI blunted and inserted into AscI, Sma sites of pLD53.SCA-E-B. Alternatively, IRES-red2 is not inserted and the Lox-tTA-Lox fragment is cloned directly into pLD53.RecA-EGFP. These shuttle vectors (pLD53-ftTA-Red-EGFP and pLD53-ftTA-EGFP plasmids) are then used as cassettes for incorporation into promoter specific BAC shuttle vectors.

Two polynucleotide sequences of upstream and downstream portions of genomic DNA, designated boxes A and B (FIG. 6), are amplified using PCR from a selected BAC promoter clone. The boxes are provided by PCR of the BAC clone of interest. The PCR primers for box A are designed so the 5′ primer incorporates an AscI site and the 3′ primer doesn't incorporate anything. The PCR product is purified and digested by AscI and inserted into a NotI-blunted AscI digested pLD53-ftTA-Red-EGFP (or pLD53-ftTA-EGFP) plasmid. The B box PCR is designed so that the 5′ primer incorporates a PacI site and the 3′ primer incorporates a FseI site. The PCR product is then purified and digested by PacI and FseI and inserted into the pLD53-ftTA-Red-EGFP (or pLD53-ftTA-EGFP) digested by the same enzymes. These vectors, containing the A box and B box specific for the promoter of interest represent the BAC shuttle vectors that are going to be used for electroporating the BAC of interest.

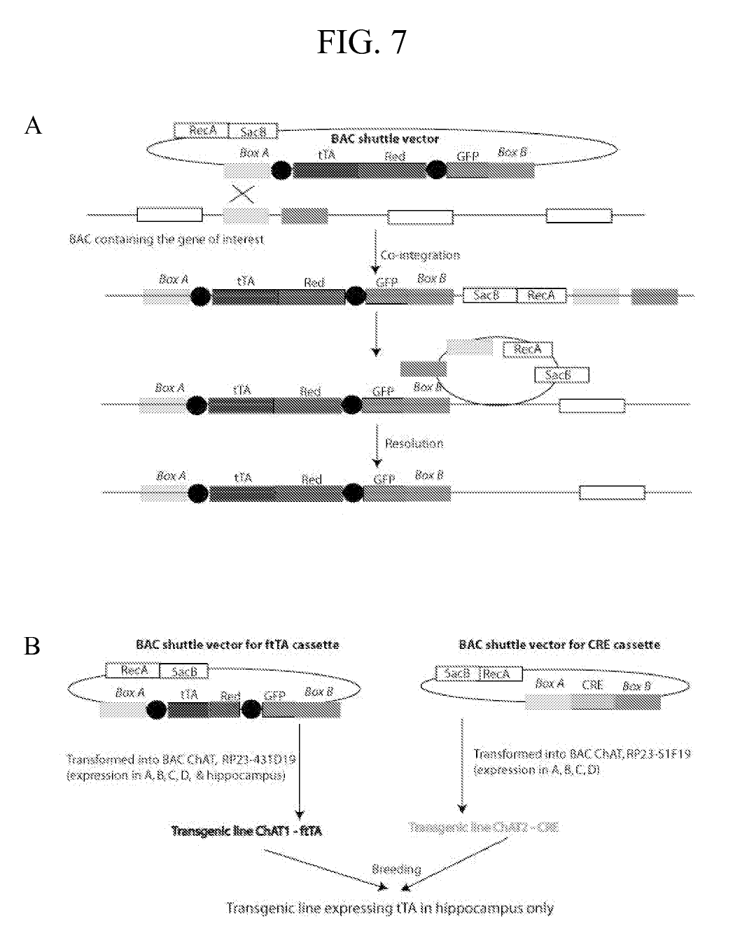

FIG. 7A illustrates the strategy for obtaining the transgenic BACs. The protocol is based on that described by the GENSAT project. In brief, the BAC shuttle vectors are electroporated into the corresponding BAC host cells. Co-integrates are selected in culture using ampicillin and chloramphenicol. The correct co-integrates are analyzed by Southern blots and then a resolution step is performed on these clones by the use of chloramphenicol and sucrose (negative selection). The BACs obtained are then purified prior to microinjection to produce transgenic animals.

When a ftTA transgenic line is crossed with a Cre expressing line, progeny are produced that express the two transgenes in an overlapping set of cells (FIG. 7B). In the example described above, the vectors are designed so that the red fluorescent protein can be detected in the transgenic animals, before removal of the ftTA and GFP (green florescence) can be detected in cells where the tTA cassette is removed after crossing with a Cre animal.

Progeny from the cross that possess both ftTA and Cre transgenes are capable of expressing a transgene in motoneurons and brainstem nuclei in a subtracted pattern (relative to either promoter), enabling the study of cholinergic neurons in the forebrain alone without disruption of essential motor functions. Such a subtractive transgenic animal is useful, for example, for producing animal models of Alzheimer disease (such as by expressing a transgene that is a silencer construct or a particular gene involved in Alzheimer disease, for example, amyloid precursor protein (APP) or a presenilin).

### Methods for Generating the ChAT-ftTA Construct

ChAT-ftTA was generated according to similar procedures as described for the Drd4 injection construct (see Example 4). Details of the methods for generating ChAT-ftTA are as follows.

Subcloning the A box into the pBSloxPtTAloxP (pBSftTA)

The A box was amplified out of the BAC RP23-246B12 using the ChATAbox5′ AscI and ChATAbox3′FseI primers. The product was purified and ligated into pBSftTA using the restriction sites AscI and FseI mutagenized by the primers into the ends. The positive clones were screened using PCR for the A box primers. The products were sequenced using the T3 (SEQ ID NO: 7) and T7 (SEQ ID NO: 8) primers.

Subcloning the AboxftTA Cassette into the Shuttling Vector

The cassette was transferred into the vector pLD53.SC-AB_MCS (a modified vector based on the pLD53.SC-AB with an inserted multiple cloning site) using the AscI and XhoI sites to construct the recombination cassette. The ligation was screened using PCR with the A box and tTA primer sets, and restriction digests. One positive clone was selected for homologous recombination after confirmation by sequencing using the RecA_{—}7023Fwd (SEQ ID NO: 9) and EGFP_{—}150Rev primers (SEQ ID NO: 10).

### Recombination

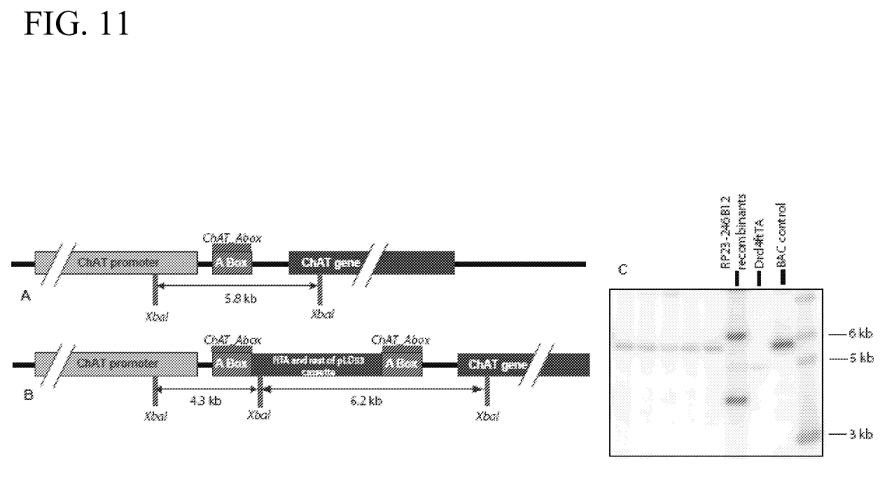

Recombination was performed according to the same protocol as described for Drd4 (see Example 4). Thirty-six colonies were picked and screened using PCR with the tTA primers. The 16 colonies exhibiting the strongest positive signal were selected and screened using Southern blotting with a radiolabeled ChAT A box probe. One of the colonies showed the expected pattern of the recombinant (see FIG. 11). The BAC was sequenced and prepared for injection.

### Preparation of Injection Construct

The injection buffer for BAC transgenesis (10 mM tris, pH 7.5, 0.1 mM EDTA, 100 mM NaCl) was prepared and filtered using a 0.2 mm filter. The DNA was diluted into the injection buffer to a concentration of 0.5 ng/μl. The diluted DNA was then purified using drop dialysis using nitrocellulose microdialysis membranes (Millipore, Inc.). The nucleotide sequence of the ChAT_ftTA injection construct is set forth as SEQ ID NO: 22.

### Example 3

### Transgene Expression in the Prefrontal Cortex

The prefrontal cortex (PFC) connects extensively with areas of the brain involved in processing external (sensory and as motor cortices) and internal (limbic and midbrain structures) information. The neurons of the PFC are activated by all types of sensory stimuli, before and during different actions, with regard to past memories, and in anticipation of future events. Neurons of the PFC are also affected by attention and motivational state (Miller and Cohen, Annu. Rev. Neurosci. 24:167-202, 2001). Therefore, the PFC has been implicated in controlling abstract information needed for intelligent behavior (Miller et al. Philos. Trans. R. Soc. Lond. B. Biol. Sci. 357(1424):1123-1136, 2002). Although the role of PFC remains largely unclear, it has been hypothesized that schizophrenia results from improper gating of information flow in the prefrontal cortex (Seamans and Yang, Prog. Neurobiol 74(1):1-58, 2004).

The principal neurons of the prefrontal cortex receive dopaminergic innervation from the midbrain ventral tegmental area. This dopaminergic innervation may be responsible for the association of tasks (and therefore rules for the task) with rewards in order to reinforce the pattern of activity responsible for attaining a goal. The dopaminergic innervation necessitates the expression of the dopamine receptor D4 (Drd4) in the principal neurons of the PFC. Expression of Drd4 in the PFC has been demonstrated (Gong et al. Nature 425(6961):917-925, 2003; Noain et al. Eur. J. Neurosci. 24(9):2429-2438, 2006), particularly in the orbital, prelimbic, cingulate and rostral agranular portions.

Using the drd4 promoter, lines of mice can be developed that express a transgene, such as a silencer construct (or other gene of interest) in pyramidal neurons (D4 dopamine receptor expressing neurons) of the prefrontal cortex. Silencers have the advantage of both spatial specificity (inactivation of only a specific neuron type, in location and function) and temporal specificity (neurons can be deactivated or ‘silenced’ during experiments only, and can be reverted back to normal afterwards). However, the drd4 promoter alone also promotes expression in retinal ganglion cells, potentially blinding the animals. Using a subtractive strategy one can subtract retinal expression simply by crossing an ftTA drd4 line to a retinal Cre line, such as Chx10_Cre. Such a cross produces progeny that are relevant animal models for normal and pathological function of the prefrontal cortex. For example, a model of the negative symptoms of schizophrenia can be produced by expressing a silencer construct in the D4 expressing neurons of prefrontal cortex.

To avoid problems associated with retinal expression, a subtractive transgenics strategy can be implemented by crossing a drd4 ftTA line with one of several available lines expressing Cre from a retina-specific promoter (such as those described by Furuta et al. Genesis. 26(2):130-2, 2000; Akimoto et al. Invest. Opthalmol. Vis. Sci. 45(1):42-7, 2004; Li et al. Genesis 41(2):73-80, 2005; as well as several produced by the GENSAT project). Progeny with both the ftTA and Cre transgenes do not express a tetO regulated transgene in the retina but retain expression of such a transgene in the prefrontal cortex, an area of enormous interest in the both cognitive and clinical sense.

### Example 4

### Drd4 Promoter Constructs for Prefrontal Cortex Studies

### BACKGROUND

### Preparation of the tTA Line

In order to achieve stable expression using a native promoter, it is advantageous to mimic physiological conditions as much as possible. The promoter elements of a particular gene are most commonly present within the 10 kb upstream region, but may be much further upstream or even downstream. Because of this, there is often variable gene expression when using minimal promoter constructs, showing mosaic patterns due to incomplete expression or leaky expression, or both. While that is useful for obtaining different patterns of expression using the same construct, it is not desirable when trying to express the construct exactly where a gene, Drd4 in this case, is expressed. Therefore, to better mimic physiological conditions, tTA can be driven by the Drd4 promoter in an environment very similar to that experienced by the Drd4 gene, such as by using a BAC-based construct (Gong et al. Nature 425(6961):917-925, 2003).

### Building the Recombination Cassette

Since the BACs are extremely large and contain multiple sites for each restriction enzyme, subcloning through restriction digests and subsequent ligation is not feasible. However, one suitable technique is homologous recombination, wherein a part of the sequence from the BAC is replicated onto the recombination cassette. In case of transfer into the BAC, two recombination sites or one recombination site can be used. In this example, one recombination site was used, termed the “A box”. For recombination into BACs, a special type of vector, called a shuttle vector, is used to carry the recombination cassette. The shuttle vector used for generating the Drd4 constructs was pLD53.SC-AB (Gong et al. Genome Res. 12(12):1992-1998, 2002, incorporated by reference herein). The recombination cassette also contains the tTA gene and loxP sites on either side of tTA. The loxP sites were designed as single stranded oligonucleotides that, when joined, would have restriction sites at the ends for subcloning. The pBluescript II KS+ (Stratagene, Inc.) was used as the basic cloning vector. The mammalianized tTA was extracted from the pTetOff plasmid (Clontech, Inc.). After inserting the loxP sites, tTA and the A box into pBluescript II KS+, the construct was excised and inserted in the shuttle vector by subcloning. The resulting recombination cassette with A box and ftTA (tTA flanked by loxP sites) was used for homologous recombination.

Homologous Recombination into BACs

The BACs contained chloramphenicol resistance and the plasmid contained ampicillin resistance. The BACs were in DH10β cells, which were made electrocompetent. Transforming the DH10β cells with purified recombination plasmid results in insertion of the plasmid into the cells. The cells were allowed to grow in medium containing both ampicillin and chloramphenicol. If the plasmid is not incorporated, the cell dies as it doesn't have any resistance to ampicillin from the BAC it contains. If the plasmid is taken into the cell, but does not recombine with the BAC, the cell survives, but does not multiply because the plasmid cannot be replicated (pLD53.SC-AB contains a R6Kγ origin of replication and therefore, cannot replicate in the pir⁻DH10β cells (Filutowicz and Rakowski, Gene 223(1-2):195-204, 1998)). The cell will only survive and multiply if the plasmid is integrated into the BAC. pLD53.SC-AB contains the RecA gene which causes highly efficient homologous recombination as shown by Gong et al. (Genome Res. 12(12):1992-1998, 2002). Integration in the presence of RecA usually occurs in the correct position (i.e., recombination should be homologous), which is downstream of the Drd4 promoter (as specified by the A box) such that the tTA is regulated by it.

### Screening for Successful Co-Integrates

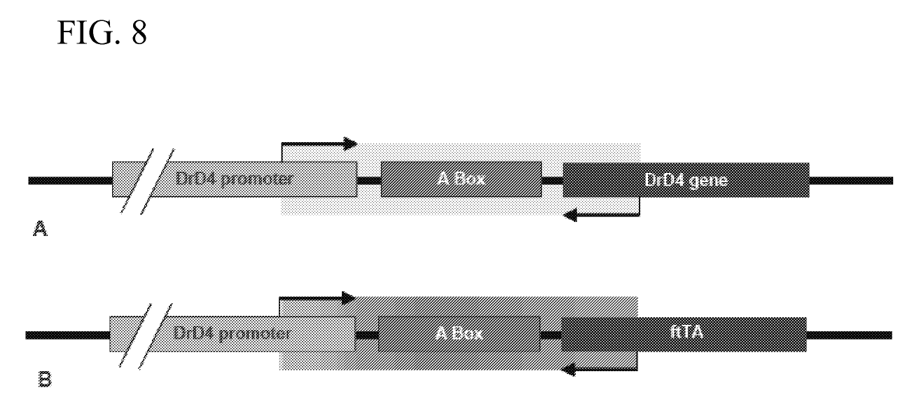

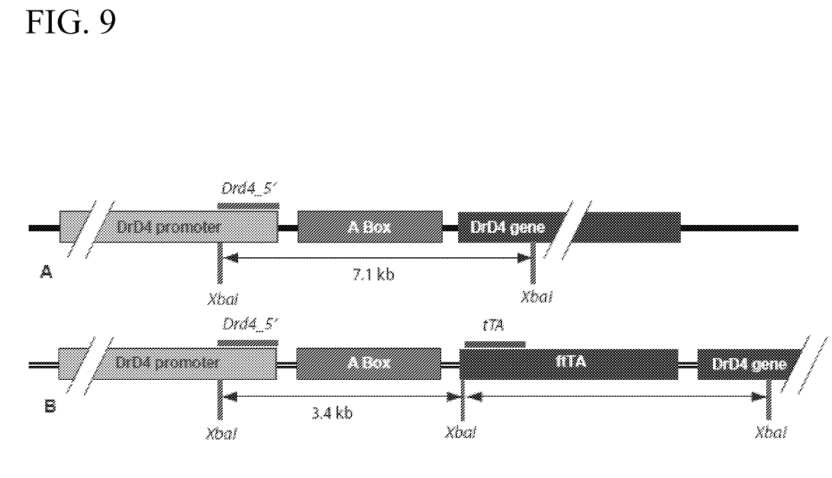

Successful co-integrates contain the Drd4 promoter, followed by the A box region, followed by the ftTA insert and part of the shuttle vector, at least up to the β-lactamase gene. Co-integrates can be identified using such techniques as PCR and Southern blot, designed to differentiate between the native and recombinant BACs. For PCR detection, primers are designed such that the forward primer is present on the BAC and the reverse primer is present either (a) on the BAC downstream of the A box sequence, or (b) on the recombination cassette downstream of the A box. The PCR using (a) will amplify only in BACs having no insert and the PCR using (b) will amplify only in BACs with the correct insert at the correct site. This is illustrated in FIG. 8. The forward primer used was Drd4_{—}5′i (SEQ ID NO: 11) and the reverse primers were Drd_gene_rev (a) (SEQ ID NO: 12) and pLD800_rev (b) (SEQ ID NO: 13). For screening by Southern blot, the probes used corresponded to an 800 bp sequence upstream of the A box (Drd4_{—}5′) and a 700 bp sequence in the tTA (tTA). The BAC DNA was digested using a restriction enzyme that cuts only upstream of the Drd4_{—}5′ sequence and some distance downstream of the 3′-terminus of the A box. The Drd4_{—}5′ probe binds to a long fragment of DNA that is specific (7142 bp) and the tTA probe will not bind at all to the native BAC. However, in case of homologous recombination, the insert is interposed in between the two sites of the restriction enzyme, and brings with itself another restriction site that cuts up the ˜7 kb band into smaller fragments. Thus, the hybridization of the Drd4_{—}5′ probe is to a smaller fragment (see FIG. 9).

### Materials & Methods

### Bacterial Strains

Three strains of E. coli were used in the study. For the replication of plasmids based on pBluescript, the DH5α strain was grown and maintained as ultracompetent stocks at −80° C. using the TSS protocol (Chung et al. Proc. Natl. Acad. Sci. U.S.A. 86(7):2172-2175, 1989). E. coli DH5α was grown in the presence of ampicillin (100 μg/ml). For plasmids based on pLD53.SC-AB, pir2 ultracompetent cells (Invitrogen, Inc.) were used and ampicillin (100 μg/ml) was used for selection. For BACs, DH10β cells containing the BAC were used. DH10β cells were grown in presence of chloramphenicol (12.5 μg/ml).

### Transformation of Bacterial Strains

The DH5α and pir2 chemically competent cells were transformed with 30 ng circular DNA and approximately 90 ng ligation product. The cells were incubated for 30 minutes, followed by addition of 900 μL of SOC. The cells were allowed to recover for 1 hr at 37° C. and 100 μl of the mixture was plated on LB plates containing ampicillin and the rest of the mixture was spun down at 10,000 rpm for 30 seconds. The supernatant was discarded except for 100 μl. The cells were resuspended and plated. The plates were incubated for 16 hours at 37° C. and examined.

The DH10β cells containing the BACs were made electrocompetent and transformed with shuttle vector pLD53.SC-AB containing the recombination cassette according to the protocol described by Gong et al. (Genome Res. 12(12):1992-1998, 2002).

### Plasmid Isolation

The alkaline lysis method was used for the isolation of plasmid for screening of colonies. Approximately 1.6 ml of a stationary phase culture was spun for 2 minutes in a microcentrifuge at 14,000 rpm at 4° C. The cell pellet was resuspended in 100 μl of resuspension buffer (25 mM Tris-Cl, 50 mM glucose, 10 mM EDTA at pH 8.0) by vortexing. To this suspension, 200 μl of lysis buffer (0.2N NaOH, 1% v/v SDS) was added and the mixture was kept at room temperature for less than 5 minutes. To the lysate, 150 μl of neutralization buffer (3M potassium acetate, glacial acetic acid, pH 5.5) was added and the mixture was kept on ice for 10 minutes. Then 450 μl 5M LiCl was added and the mixture was kept on ice for 10 minutes. The cell debris containing chromosomal DNA was precipitated by spinning the suspension for 10 minutes in a microcentrifuge at 14,000 rpm. The supernatant containing plasmid DNA was precipitated with 650 μl isopropanol and then with 1 ml 70% ethanol. The final pellet was dissolved in 50 μl water.

For growing up plasmid stocks and for subsequent sequencing and ligation steps, plasmid was prepped from 100 ml of overnight cultures (incubated at 37° C.) using Qiagen Midiprep kit according to the manufacturer's protocol. The DNA was resuspended in distilled water.

### Restriction Endonuclease Digestion of DNA Fragments and Cloning in Plasmid Vectors

Restriction digests for preparing insert and vector for ligations were performed using endonucleases (New England Biolabs, Inc.) according to the manufacturer's protocol. DNA was digested in a total volume of 100 μl for 2 hours. Restriction digests for screening of colonies were performed using variable amounts of plasmid prep (100-500 ng) in a volume of 20 μl for 1 hour.

### Visualization of DNA Bands Using Agarose Gel Electrophoresis

The plasmid DNA following restriction digestion, and before ligation, was visualized on 0.8% agarose gels using Tris-acetate-EDTA buffer, with 0.5 μg/ml ethidium bromide, and using 0.5 ng 1 kb DNA ladder (New England Biolabs, Inc.). The BAC DNA was visually compared to a λ-HindIII ladder (New England Biolabs, Inc.). The PCR products were compared using a 100 bp ladder (New England Biolabs, Inc.) on a 1% agarose gel. The DNA was visualized and quantified using the Gel documentation system (Biorad Laboratories, Inc) by comparison with the DNA ladder.

Extraction of DNA from Agarose Gel

The agarose pieces containing DNA bands were excised under long-wave UV illumination. The agarose blocks were run in a perpendicular direction in the same buffer in dialysis bags to run the DNA off the gel, and the DNA was extracted from the buffer using Elutip-D minicolumns (Schleicher & Schuell, Inc.), according to the manufacturer's protocol. The eluate in high-salt buffer was then precipitated with ethanol.

TABLE 1
Primer and Oligonucleotide Sequences

| | | | |
|---|---|---|---|
| | | SEQ | |
| Primer Name | Sequence* | ID NO: | Application |

| | | | |
|---|---|---|---|
| DrdABox5′AscI | CAGCTAGCAGGGCGCGCCGCACTGA | 1 | Primers used for the |
| | CTGATGGAGACTTGGGAAGAGAG | | amplification of the A box |
| DrdABox3′FseI | GAATTCGGCCGGCCTGCCACTGCTG | 2 | fragment from the BAC |
| | AACCCGCTCCGGGAGGCGC | | vector RP-23 1 34L4 |
| loxP 1 | CACGGCGCGCCTAGGCCGGCCATAA | 3 | The two oligonucleotides |
| | CTTCGTATAGCATACATTATACGAA | | annealed together were used |
| | GTTATGTTTAAACACCCTGCAGGAG | | for the construction of the 5′ |
| | GGGCCCACA | | loxP site along with |
| loxP1bis | AGCTTGTGGGCCCCTCCTGCAGGGTG | 4 | restriction sites to facilitate |
| | TTTAAACATAACTTCGTATAATGTA | | cloning |
| | TGCTATACGAAGTTATGGCCGGCCT | | |
| | AGGCGCGCCGTGGTAC | | |
| loxP2 | ATCAGACTAGTACGCGATCGCTAAT | 5 | The two oligonucleotides |
| | AACTTCGTATAGCATACATTATACG | | annealed together were used |
| | AAGTTATATTTAAATACCTCGAGAG | | for the construction of the 3′ |
| | GCGGCCGCATG | | loxP site along with |
| loxP2bis | AATTCATGCGGCCGCCTCTCGACGTA | 6 | restriction sites to facilitate |
| | TTTAAATATAACTTCGTATAATGTA | | cloning |
| | TGCTATACGAAGTTATTAGCGATCG | | |
| | CGTACTAGTCTGAT | | |
| T3 | AATTAACCCTCACTAAAGGG | 7 | Primers used for screening |
| T7 | GTAATACGACTCACTATAGGGCG | 8 | and sequencing inserts |
| | | | subcloned into pBluescript II |
| | | | KS+ |
| RecA_7023fwd | CGAAAACGTGGTGGGTAGCGAAACC | 9 | Primers used for screening |
| | CGCG | | and sequencing inserts |
| FGFP_150rev | CGCCCTCGCCGGACACGCTGAAC | 10 | subcloned into pLD53.SC- |
| | | | AB |
| Drd4_5′i | GACTGGGCCTTGGAGGTGCC | 11 | Primers used for screening of |
| Drd4_gene_rev | CAGCCAGGCTCACGATGAAG | 12 | recombinants by PCR from |
| pLD800_rev | CACCTAGCTTCTGGGCGAGTTTACG | 13 | native BAC |

*The bases in bold are the actual sequences cloned within the flanking restriction sites

TABLE 2
Cloning vectors and recombinant plasmids

| Vector | Description | Source |
|---|---|---|
| pBluescript II KS+ | Basic cloning vector containing multiple cloning site and | Stratagene, Inc. |
| | galactose gene for blue-white selection. | |
| pTetOff | Source of mammalianized tetracycline trans-activator | Clontech, Inc. |
| | (tTA) in the construct. | |
| pLD53.SC-AB | Shuttle vector containing RecA gene for high-efficiency | Dr Nathaniel Heintz, |
| | homologous recombination into BACs and R6kγ origin | Rockefeller |
| | of recombination that necessitates pir gene to be | University |
| | produced by the host bacteria for replication. | |
| RP-23 134L4 | BAC initially used for homologous recombination (see | Children's Hospital |
| | Gong et al., 2003). | Oakland Research |
| RP-23 320N24 | BAC used for the final successful homologous | Institute (CHORI) |
| | recombination. Both the lines of BAC are from the BAC | |
| | DNA library of mouse genome as constructed by Pieter | |
| | de Jong's lab at CHORI. | |

### Ligation of DNA Fragments

The ligations into plasmid vectors were performed using T4 DNA ligase (New England Biolabs, Inc.) using 150 fM of insert and 30 fM of vector DNA. Ligation mixtures were incubated in a volume of 20 μl overnight in a 16° C. bath. Half the volume of ligants was transformed the next day and simultaneously replica-plated, inoculated in minicultures and screened using PCR amplification.

### Polymerase Chain Reaction (PCR)

For screening, PCR was carried out in a total volume of 25 μl in 1× Thermopol buffer (New England Biolabs), containing 20 to 50 ng template DNA, 25 pM of each primer, 0.2 mM of each dNTP (Fermentas, Inc.), and 1 μl of Taq polymerase in a thermocycler (icycler, Biorad Laboratories, Inc). The amplification of A box fragments was performed using Herculase hi-fidelity polymerase (Stratagene, Inc.).

### DNA Sequencing

For direct sequencing of the plasmid DNA, 300-600 ng template was combined with 7 pM primer and the volume made up to 13 μl. The sequencing reaction was carried out in MJ 4-engine thermocyclers and the samples were analyzed in the 3130XL 16-capillary Genetic Analyzer (Applied Biosystems, Inc.). For sequencing of BAC DNA, 1 μg template DNA was provided.

### Dot-Blot Hybridization for Screening Successful Colonies

For screening positive ligants following the ligation of the A box ftTA cassette from cloning vector to shuttle vector, radioactive colony lifts were used. From the original plate containing transformants, discrete colonies were replica-plated onto twin 15 cm-diameter plates of LB agar containing 12.5 μg/ml chloramphenicol. Colonies were directly plated onto one plate, and over a nylon membrane (Hybond+ circular nylon membrane, Amersham Biosciences plc) on the other plate. The nylon membrane was then denatured using 0.5 N NaOH and 1.5 M NaCl solution and neutralized using 0.5 M Tris-Cl and 1.5 M NaCl solution. Proteins were removed by incubation with Proteinase K solution, followed by a wash in the neutralization solution, and baked at 80° C. for 1 hour to bind DNA. The membrane was hybridized to a radio-labeled (dCTP³²) tTA probe and finally read in a STORM Phosphorimager (GE Healthcare Life Sciences, Inc.). The replicates of the colonies successfully binding the probes were analyzed using restriction digests. One colony was selected.

For screening positive co-integrates following the first attempt of homologous recombination of BACs, the colonies were replica-plated as above, but hybridized to a Riboprobe processed by transcribing the antisense RNA to A box ftTA sequence as in pBSftTA. The DIG nonradioactive kit for random-primed labeling of riboprobes (Roche Diagnostics Corp., Inc.) was used according to the manufacturer's protocol.

### Preparation of BAC DNA

BAC DNA was prepped from overnight cultures of 5 ml using modified alkaline lysis protocol (Sambrook & Russell, Molecular Cloning: A Laboratory Manual. Cold Spring Harbor, N.Y.: Cold Spring Harbor Laboratory Press, 2001) for the purpose of Southern blot and PCR to characterize possible recombinants. For the purpose of DNA sequencing to confirm recombinants and for the preparation of injection constructs, the Nucleobond plasmid midi-kit (Clontech, Inc.) was used with twice the volumes of buffers used as compared to manufacturer's protocol for BAC/PAC and low-copy vectors, and was eluted in 200 μl water.

### Southern Blot for Characterization of Recombinants

The BAC DNA was digested using restriction endonuclease XbaI (New England Biolabs, Inc.) and run on a 0.8% agarose gel using Tris-acetate-EDTA buffer, with 0.5 μg/ml ethidium bromide and using 0.5 ng 1 kb DNA ladder and λ-HindIII ladder (New England Biolabs, Inc.). The gel was run at 90 volts in a submarine gel electrophoresis device (Biorad Laboratories, Inc) and was depurinated with 0.25 N hydrochloric acid and denatured using 0.5 N NaOH and 1.5 M NaCl solution, and neutralized using 0.5 M Tris-Cl and 1.5 M NaCl solution. The DNA was transferred onto nylon membrane (Hybond XL nylon membrane, Amersham Biosciences plc) using wet transfer in 20×SSC. The blot was crosslinked using a stratalinker and hybridized using the nonradioactive DIG-labeled kit (Roche Diagnostics Corp.) using random-primed probe. Bands were detected using CSPD chromogenic detection kit (Roche Diagnostics Corp.) and fluorescence was recorded on Kodak X-OMAT Blue autoradiography film (Perkin Elmer, Inc.) using exposures of 5, 15 and 30 minutes.

### Results

Subcloning loxP1 into pBluescript II KS+ (pBS)

The loxP1 oligonucleotide dimer was constructed by mixing loxP1 and loxP1 bis single stranded oligonucleotides and boiling them together to denature and re-anneal by gradual cooling to room temperature. LoxP1 was subcloned using the restriction sites KpnI and HindIII that had been designed into the oligonucleotide and inserting loxP 1 into the KpnI and HindIII sites of pBS. The potentially positive clones were screened using the restriction sites AhdI and AscI on the vector pBS, and the insert loxP1, respectively. The positive clone was confirmed by direct sequencing using the T3 and T7 primer sites flanking the cloning region on pBS.

Subcloning tTA into the pBSloxP

The tTA was excised out of the pTetOff using the EcoRI and BamHI sites and put into pBSloxP using those sites. The ligation product was transformed and potential positive clones were screened using the EcoRI/BamHI digest that liberates the insert and the AhdI and SnaBI sites present on the vector and the insert respectively. The clone was confirmed using the T3 and T7 sites.

Subcloning loxP2 into pBSloxPtTA

The loxP2 oligonucleotide dimer was constructed from loxP2 and loxP2bis single stranded oligonucleotides. The oligonucleotide was ligated using the NotI and SpeI sites designed into it and on the 3′ end of the tTA on the pBSloxPtTA vector. The ligation was screened by double digests using the XhoI and PmeI sites and the XhoI and ApaI sites. The ligation was screened using restriction digests and sequenced using T3 and T7 primers.

Subcloning the A box into the pBSloxPtTAloxP (pBSftTA)

The A box was amplified out of the BAC RP23-134L4 using the DrdAbox5′AscI (SEQ ID NO: 1) and DrdAbox3′FseI primers (SEQ ID NO: 2). The product was purified and ligated into pBSftTA using the restriction sites AscI and FseI mutagenized by the primers into the ends. The positive clones were screened using PCR for the A box primers and by getting a positive signal. The products were sequenced using the T3 and T7 primers.

Subcloning the AboxftTA Cassette into the Shuttling Vector

The cassette was transferred into the vector pLD53.SC-AB using the AscI and SwaI sites to construct the recombination cassette. The ligation was screened using colony lifts (see Materials and Methods), and restriction digests with FseI, XbaI, and NcoI, which have sites both in the insert and vector. One positive clone was selected for homologous recombination after confirmation by sequencing using the RecA_{—}7023Fwd (SEQ ID NO: 9) and EGFP_{—}150Rev (SEQ ID NO: 10) primers.

Homologous Recombination into BAC

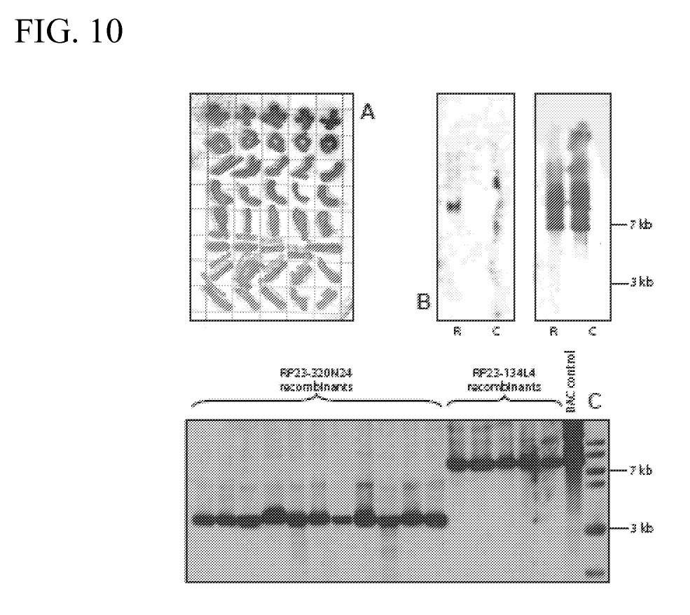

The BAC initially used for recombination was RP23-134L4, as described by Gong et al. (Nature 425(6961):917-925, 2003). The BAC was made competent and transformed. The inoculum was screened using serial dilutions in double selection medium, and the colonies that grew in a plate with Chlor/Amp were selected for further screening. The colonies were screened using dot-blot with tTA riboprobe, and all 40 colonies were positive for the insert (FIG. 10A). They were screened using two sets of PCR reactions. However, all of the clones were positive for both. The PCR reaction specific for the recombinant was sequenced, and the run read all the way from the BAC to the insert, demonstrating homologous recombination. But as both the PCR reactions were positive, it meant that though the recombination cassette had been incorporated correctly there was heterogeneity of BACs in the cell, essentially a mixture of BACs, some native and some recombinant. To determine the proportion of native versus recombinant, the clones were screened using Southern blot for both tTA and Drd5′probe, as shown in FIG. 9. The potential recombinant bound to tTA (unlike the original BAC), which meant there was incorporation of insert, but the Drd5′ probe bound significantly more to the 7 kb band (native) compared to 3 kb (recombinant), as shown in FIG. 10B. The blot showed that the number of copies of native BAC in the mix was much more than the recombinant.

The next procedure used both RP23-134L4 and RP23-320N24 strains. They both contain largely overlapping parts of the mouse genome, and need the same A box sequence. Recombination was performed according to the same protocol and 48 colonies, 24 of RP23-134L4 and 24 of RP23-320N24, were picked. They were screened using double-PCR using same strategy as described herein. The colonies that exhibited positive amplification for recombinant reaction were selected. Of the RP23-134L4 colonies, none of the clones were fully negative for the native BAC reaction; thus those with fainter bands were picked. Sixteen clones in total were selected and screened using nonradioactive (DIG-labeled) Southern blot. The positive control was pLD53AboxftTA recombination vector, and the negative control was the native 320N24 BAC. The blot was hybridized to Drd4_{—}5′ probe. All of the 320N24 clones showed 3.4 kb bands (i.e., contain recombinants only), while none of the 134L4 clones showed recombination. Clone 13 was chosen for injection and was grown up and purified using the Nucleobond kit. The DNA was run in a gel and quantified against the λ-HindIII marker, and checked for absence of genomic DNA or RNA, and selected for preparation of injection construct.

### Preparation of Injection Construct

The injection buffer for BAC transgenesis (10 mM tris, pH 7.5, 0.1 mM EDTA, 100 mM NaCl) was prepared and filtered using a 0.2 mm filter. The DNA was diluted into the injection buffer to a concentration of 2 ng/μl. The diluted DNA was then purified using drop dialysis using nitrocellulose microdialysis membranes (Millipore, Inc.), and the purified DNA was diluted in equal volume of 2× polyamine solution (1/500 dilution of 30 mM Spermine tetrahydrochloride and 70 mM Spermidine trihydrochloride (Sigma Laboratories, Inc.)). A final concentration of 0.25 ng/μl diluted only in injection buffer was used for injection. The nucleotide sequence of the Drd4_ftTA injection construct is set forth as SEQ ID NO: 23.

### Example 5

### Expression in Subsets of Inhibitory Interneurons

Inhibitory interneurons are well-suited for a subtractive approach, not only because of their clear clinical relevance (for example, epilepsy and hypnotic drugs) and the central role they play in neural circuits, but also because they have been extensively characterized cytologically, leading to a classification of interneurons based upon which calcium binding proteins and neuropeptides they express, as well as their cytoarchitecture. This extensive molecular characterization simplifies selection of various combinations of promoters and facilitates production of subtractive transgenic animals that express in particular subsets of inhibitory interneurons. For example, while all inhibitory interneurons in the CNS are GABAergic, around half of them also express the Ca++ binding protein parvalbumin (parv), while the others express other calcium binding proteins such as calbindin or calretinin. Of these, some of them express neuropeptide Y (NPY), others express somatostatin (SMT), yet others express cholecystokinin (CCK), and others express vasoactive intestinal peptide (VIP). However, they all express only one calcium binding protein and one, or sometimes two, neuropeptides.

The neuropeptides and calcium binding proteins are expressed in other cells as well, limiting the utility of a direct approach. Therefore, the modularity and flexibility of the subtractive approach offers benefits over direct transgenic procedures for the manipulation of inhibitory interneurons. In addition, the subtractive transgenic approach can be combined with complementary approaches (such as the dual-recombinase approach described by Awatramani et al., Nature Genet. 35(1):70-75, 2003).

For example, a Cre recombinase line under the control of the GAD 65 promoter expresses Cre in most, if not all, inhibitory interneurons. Flanking it with FRT sites enables its use in the intersectional approach described by Awatramani et al. (Nat. Genet. 35(1):70-75), which describes a dual recombinase-responsive indicator allele that marks cells and their descendant lineages only if they have expressed both Cre and Flp, creating an “intersectional” strategy that labels cells. The dual-recombinase approach is complementary to and can be used in conjunction with the subtractive transgenic methods disclosed herein. Making lines with the various neuropeptide and Ca²⁺ binding protein promoters driving the expression of different recombinases and inducible transactivators with recombinase sites enables the complete molecular dissection of all subclasses of interneuron. Furthermore, the resulting subtracted lines can be mated to any tetO (or any other inducible promoter) lines expressing a wide variety of transgenes without requiring the generation of novel lines. Essentially any promoter that expresses in a subset of inhibitory motorneurons can be used to drive expression of an fSTOP-tTA transgene. For example, in one combination a GAD-65_FRT-Cre-FRT line is crossed with a SMT_fSTOP_tTA line, which would specify tTA expression (and thereby transgene expression) only in SMT+ interneurons. Further specificity can be obtained by mating this line to a line expressing flp recombinase by the calbindin promoter, further restricting transgene expression to SMT+/CBP+ positive interneurons.

### Calbindin Promoter Constructs for Interneuron Studies

The calbindin promoter was inserted into pBSIIKS+ from a BAC as follows. BAC RP23 12N24 and pBSIIKS+ were digested with SacII and EcoRV. A 7.3 kb portion of the calbindin promoter directly upstream of the translation start was liberated from the BAC. The pBS_CALB construct was then used to clone the Calbindin promoter into three cassettes.

The calbindin promoter was cut out of pBS_CALB with BssHII and EcoRV and the 7.3 kb band was gel purified (Qiagen) for insertion into both the tTA and the fSTOPtTA cassette plasmids. Both cassettes were cut with KpnI, blunted with Klenow and cut a second time with AscI. Colonies were screened by PCR for the calbindin A box and positive colonies were confirmed by restriction enzyme analysis. The final injection construct was liberated with BssSI/NotI and gel purified with GeneClean.

To clone the calbindin promoter into the Cre (no GFP) cassette plasmid, pBS_CALB was cut with EcoRV and SacII and the 7.3 kb band was gel purified (GeneClean). Cre was cut with Ecl136II and SacII. Colonies were screened by PCR for the calbindin A box and positive colonies were confirmed by restriction enzyme analysis. The final injection construct was liberated with BssSI/DraIII and gel purified with GeneClean.

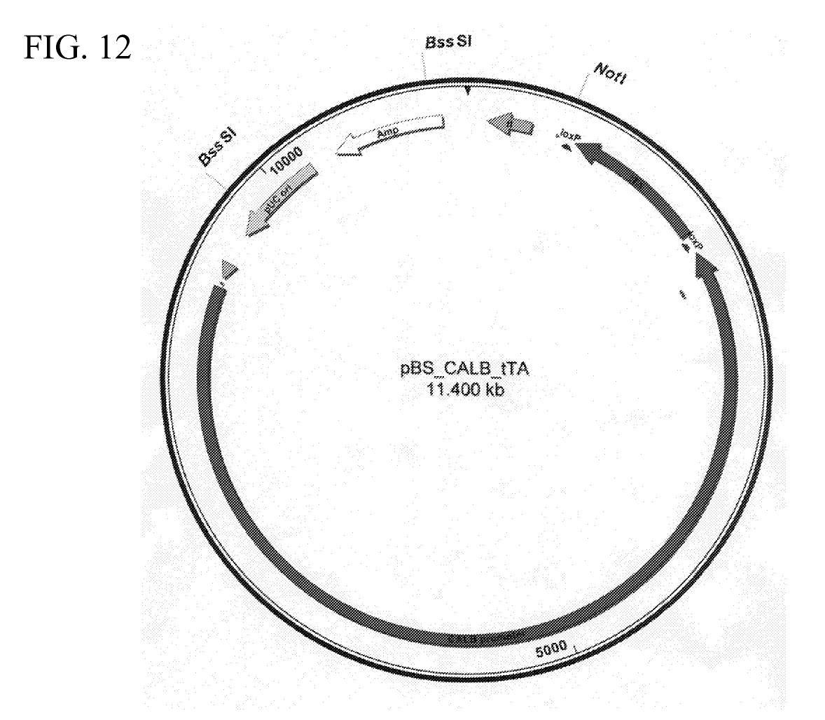

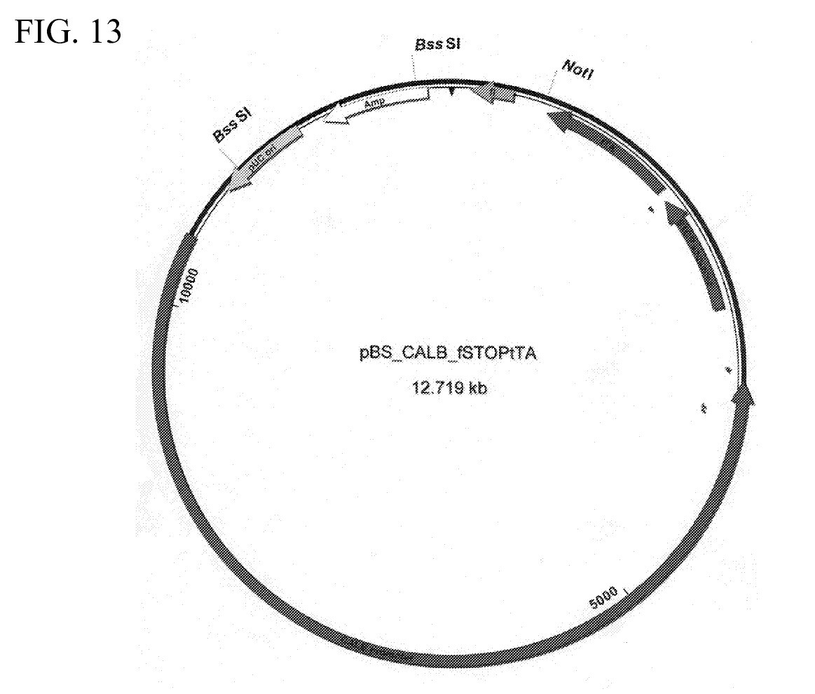

The nucleotide sequences for the calbindin injection constructs are set forth as SEQ ID NO: 24 (pBS_CALB_tTA) and SEQ ID NO: 25 (pBS_CALB_fSTOPtTA). Maps of the calbindin constructs are shown in FIG. 12 and FIG. 13.

### CCK Promoter Constructs for Interneuron Studies

The cholecystokinin (CCK) promoter was cloned by PCR using BAC RP23-234117 as a template. PCR was performed by cycling with temperature gradient annealing (95° C. for 2 minutes; 94° C. for 30 minutes; 48-62° C. for 30 minutes; 72° C. for 6 minutes; repeat the previous steps 27×; 72° C. for 7 minutes; and store at 10° C.). Table 3 below provides the sequences for the primers used to amplify segment A (just 5′ of the ATG; 2089 bp), segment B (middle segment; 5678 bp) and segment C (5′-most segment; 2488 bp). Herculase was used to amplify fragments B and C and Pfu Turbo was used to amplify fragment A.

TABLE 3
Primers for CCK promoter cloning

| | | SEQ ID |
|---|---|---|
| Segment | Primer Sequence | NO: |
| A | AATAGGCCGGCCTCAGCGTGCTCCAGCC | 14 |
| A | CCTTTCTCCATTCACCCTAGCTTG | 15 |
| B | CCTGACCTCCTTAACCACCAGGC | 16 |
| B | CCTTGCTGCTAGCTTGTAACTTGG | 17 |
| C | AAATGGCGCGCCATTGGTGAAGGAAGACACTGAGC | 18 |
| C | AAATGGCGCGCCATTGGTGAAGGAAGACACTGAGC | 19 |

The oligonucleotide sequences used for modification of the multiple cloning site of pBS KS+ were 5′-aattcggcgcgccaaaacctgcaggagctagcaaaaggccggcca-3′ (SEQ ID NO: 20) and 5′-agcttggccggccttttgctagctcctgcaggttttggcgcgccg-3′ (SEQ ID NO: 21). Fragments were cloned into the modified pBS (pCCK) with the following restriction enzymes: Fse1 and Nhe1 (fragment A); Sbf1 and Nhe1 (fragment B); and Asc1 and Sbf1 (fragment C). The CCK promoter was subcloned from the assembled pCCK CBA construct into the lox-STOP-lox tTA cassette as well as the lox-tTA-lox cassette containing plasmids by utilizing the Fse1 and Asc1 sites of those plasmids to introduce the CCK promoter upstream of the tTA genes. The injection constructs were excised from the promoter/cassette plasmid by digestion with Asc1 and Spe1.

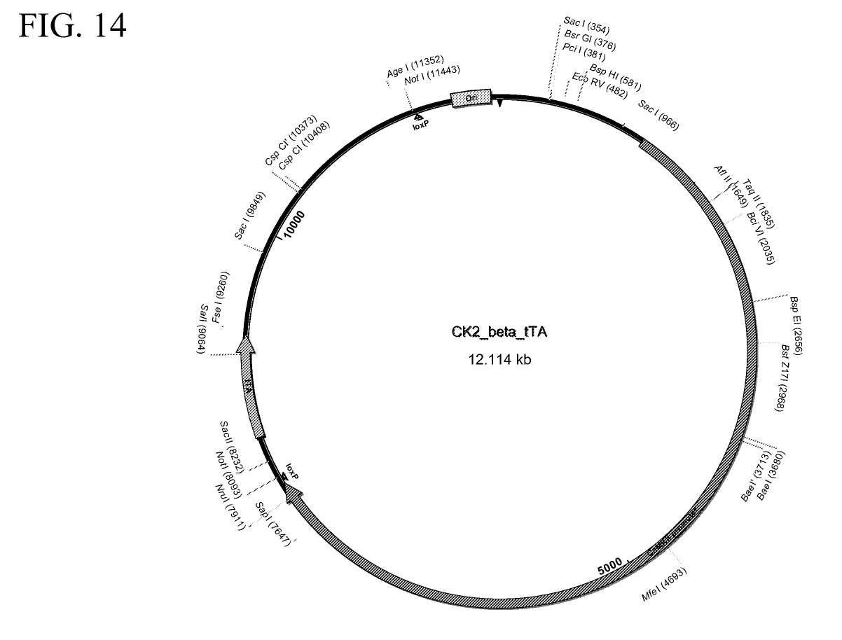

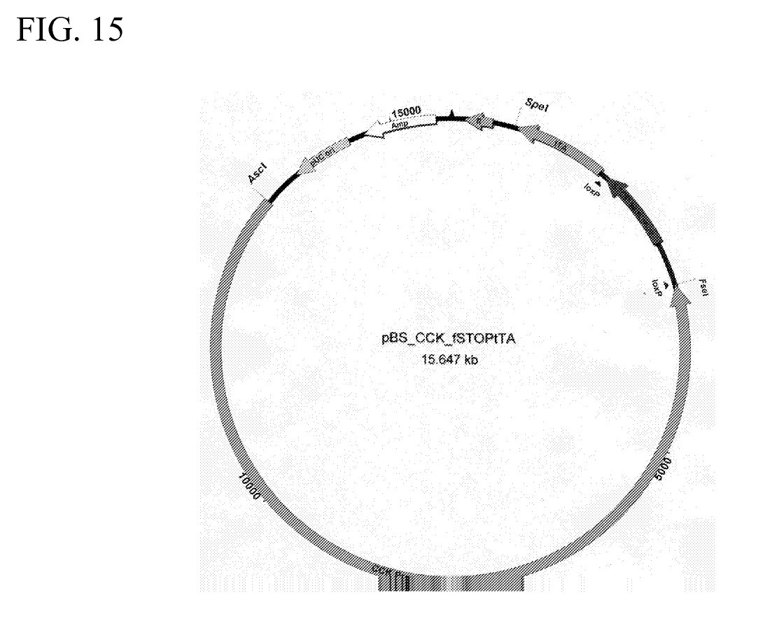

The nucleotide sequences of the CCK injection constructs are set forth as SEQ ID NO: 26 (pBS_CCK_ftTA) and SEQ ID NO: 27 (pBS_CCK_fSTOPtTA). Maps of the CCK constructs are shown in FIG. 14 and FIG. 15.

### Example 6

### Broader Application of Subtractive Transgenics

While the subtractive technique is primarily described herein with respect to the nervous system, it is readily applicable to any other functional and/or structural system in the body. For example, the methods disclosed herein can be used to increase anatomical control of transgenes within the immune system. The cells of the immune system are intrinsically suitable for a subtractive approach. Although lymphocytes are cytologically similar, they have long been characterized immunologically by the antigens they express on their cell surface, the CD (Cluster of Differentiation) antigens. The type of lymphocyte (and its maturity) correlates with the set of CD antigens on its membrane. As thymocytes mature, they begin to express various antigens that specify cell type (for example, CD3 and CD4 or CD8 for T lymphocytes), with more and more specific sets of antigens corresponding to more and more specific subsets of lymphocytes. The number of known CD antigens is approaching 300, but while in the vast majority of cases the genes encoding the antigens have been cloned, there have been very few transgenic lines made with their promoters, even though specifying transgene expression to functional subsets of lymphocytes would be of great general interest. This is mostly due to the fact that individual CD antigen promoters express in several cell types; it is the set of CD antigens that specify subtypes. Thus, a transgenic line made with a single promoter typically does not approach the necessary anatomical specificity.

For example, while all T lymphocytes express the CD5 antigen, only a small subset of B cells does. This CD5⁺ subset of B cells, sometimes called B1 cells, is involved with a more general defense against bacterial polysaccharides than the specific antibody response requiring helper T cells. Using a subtractive transgenic approach, it is possible to cleanly specify transgene expression exclusively to this subset of B cells by removing T cell expression. For instance, Cre recombinase can be expressed using the T-cell specific CD3 promoter and mice expressing this construct can be crossed with an ftTA line under the control of the CD5 promoter. Progeny expressing both transgenes are capable of expressing a transgene only in B1 lymphocytes.

### Example 7

### Subtractive Transgenics Using Viral Vectors

The subtractive transgenics strategy can also be adapted for use in animals (without the need for generating transgenic animals), including humans, by introducing the system components via viral vectors. A variety of viral vectors suitable for the methods described herein are well known in the art, including, but not limited to retroviruses, lentiviruses, adenoviruses, adeno-associated viruses and herpesviruses. For example, disease-specific biomarker information obtained using genetic or immunological methods can be used to develop disease-specific subtractive sets of viral vectors that result in regulated expression of a particular transgene (for example, a transgene encoding a marker protein, a therapeutic protein, a wild-type protein, a toxin or an effector protein, such as an apoptosis-inducing protein) only in target cells (such as diseased cells, including tumor cells, infected cells or cells comprising genetic mutations).

To accomplish subtractive transgenics in animals, three recombinant viral vectors are used: a transactivator virus (encoding a transactivator such as tTA, or a transcription factor) comprising recombinase recognition sites; a virus encoding the appropriate recombinase; and a virus expressing the desired effector transgene, wherein expression of the transgene is regulated by the appropriate responsive element (for example, tetO) or transcription factor binding site.

This system can be used to deliver any number of different types of transgenes. The transgene can encode a marker protein, such as green fluorescent protein. The transgene can also be a therapeutic transgene. For example, in the case of a disease caused by expression of a mutated protein, a therapeutic transgene can encode a wild-type version of the protein to prevent, treat or ameliorate the disease. Alternatively, the transgene can encode a toxin or other silencer if cell death of the targeted cells (such as a tumor cell) is desired.

The subtractive transgenics system can also be used to specifically allow the production of lytic virus in a particular subset of cells (for example, tumor cells). In this case, the viral vector encoding the transgene encodes the viral genes necessary for packaging of an infectious virus, essentially serving as a regulated helper virus. The lytic virus would not only be able to destroy the target cell, but would inoculate other cells or tissue, eliminating the need to re-administer this viral vector. The virus is replication-competent only in the target cell and only in the presence or absence of the regulator (such as doxycycline), depending on the particular system used.

### Example 8

### Use of Subtractive Transgenics to Treat Acute Myelogenous Leukemia

The neoplastic stem cells that cause acute myelogenous leukemia (AML) have been well characterized by flow cytometric analysis (Jordan et al. Leukemia 14(10):1777-1784, 2000; Blair et al. Blood 89(9):3104-3112, 1997; Wozniak and Kopec-Szlezak, Cytometry B Clin. Cytom. 58(1):9-16, 2004). These leukemic stem cells are serologically unique, as evidenced by the expression of an antigen that hematopoietic stem cells usually do not express (CD123), and lack of expression of some antigens hematopoietic stem cells usually do express (CD90 and CD117). This pattern of expression (expressing some, but not other antigens) is particularly well suited to the subtractive transgenics approach.

To specifically target AML stem cells, one can for example, clone the promoter for CD123 and the promoter for CD90 and use the two different promoters with the transactivator vector and the recombinase vector. For example, to selectively express a transgene in AML stem cells, the goal is to express the transgene in CD123-expressing cells (CD123⁺) AND NOT in CD90-expressing cells (CD90⁺). To accomplish this, the recombinase vector comprises the CD90 promoter and the transactivator virus comprises the CD123 promoter. In addition, the transactivator is flanked by recombinase recognition sites. Since the recombinase is expressed in all CD90⁺ cells, in any CD90⁺/CD123⁺ cells, the recombinase will excise the transactivator, rendering it non-functional. However, in CD123⁺, CD90⁻ cells, the transactivator will be expressed. The third viral vector encodes the selected transgene, which is regulated by the appropriate responsive element. Thus, the transgene is expressed only in CD123⁺, CD90⁻ cells.

This disclosure provides methods for highly selective expression of a transgene in animals. The disclosure further provides non-human transgenic animals exhibiting highly selective expression of a transgene and methods of generating such animals. It will be apparent that the precise details of the methods described may be varied or modified without departing from the spirit of the described subject matter. We claim all such modifications and variations that fall within the scope and spirit of the claims below.

1. A transgenic non-human animal comprising:
(a) a first heterologous nucleic acid encoding a recombinase operably linked to a first selective promoter;
(b) a second heterologous nucleic acid encoding a transcription factor operably linked to a second selective promoter, wherein expression of the transcription factor is regulated by expression of the recombinase; and
(c) a third heterologous nucleic acid comprising a transgene under the transcriptional control of a response element specific for the transcription factor.

2. The transgenic non-human animal of claim 1, wherein the recombinase is expressed in a first population of cells and the transcription factor is expressed in a second population of cells, wherein the first and second populations of cells comprise at least one overlapping sub-population of cells and at least one non-overlapping sub-population of cells.

3. The transgenic non-human animal of claim 2, wherein the transgene is expressed only in the at least one overlapping sub-population of cells.

4. The transgenic non-human animal of claim 2, wherein the transgene is expressed only in the at least one non-overlapping sub-population of cells.

5. The transgenic non-human animal of claim 1, wherein expression of the transcription factor is disabled by expression of the recombinase.

6. The transgenic non-human animal of claim 5, wherein the second heterologous nucleic acid comprises recombinase recognition sites flanking the transcription factor, such that expression of the transcription factor is disabled by excision of the transcription factor by the recombinase.

7. The transgenic non-human animal of claim 1, wherein expression of the transcription factor is enabled by expression of the recombinase.

8. The transgenic non-human animal of claim 7, wherein the second heterologous nucleic acid comprises a transcriptional STOP signal preceding the transcription factor that prevents expression of the transcription factor, wherein the transcriptional STOP signal is flanked by recombinase recognition sites, such that expression of the transcription factor is enabled by excision of the transcriptional STOP signal by the recombinase.

9. The transgenic non-human animal of claim 1, wherein the recombinase is a Cre recombinase, a flp recombinase or a β-recombinase.

10. The transgenic non-human animal of claim 1, wherein the transcription factor is a tTA transcription factor or a fusion protein thereof.

11. The transgenic non-human animal of claim 1, wherein the response element is a tetO transcription response element.

12. The transgenic non-human animal of claim 1, wherein at least one of the first and second selective promoters comprises a tissue specific promoter.

13. The transgenic non-human animal of claim 1, wherein at least one of the first and second selective promoters comprises a temporally specific promoter.

14. The transgenic non-human animal of claim 1, wherein at least one of the first and second selective promoters comprises an inducible promoter.

15. The transgenic non-human animal of claim 1, wherein at least one of the first and second selective promoters comprises a promoter that is selective for neural cells.

16. An isolated transgenic cell from the transgenic non-human animal of claim 1.

17. A method of producing a non-human animal that expresses a transgene in a selected population of cells, the method comprising:
identifying at least one progeny of a cross between
(a) a first transgenic non-human animal comprising first heterologous nucleic acid encoding a recombinase operably linked to a first selective promoter; and
(b) a second transgenic non-human animal comprising a second heterologous nucleic acid comprising a polynucleotide sequence that encodes a transcription factor operably linked to a second selective promoter, wherein the polynucleotide sequence is
(i) flanked by recombinase recognition sites; or
(ii) preceded by a transcription STOP signal flanked by recombinase recognition sites,
wherein the at least one identified progeny comprises the first and second heterologous nucleic acids and wherein the at least one progeny further comprises a transgene under the transcription regulatory control of a response element specific for the transcription factor.

18. The method of claim 17, wherein the recombinase is expressed in a first population of cells and the transcription factor is expressed in a second population of cells, the first and second populations of cells comprising at least one overlapping sub-population of cells.

19. The method of claim 17, wherein expression of the transcription factor is dependent on expression of the recombinase.

20. The method of claim 19, wherein expression of the transcription factor is enabled by expression of the recombinase.

21. The method of claim 19, wherein the expression of the transcription factor is disabled by expression of the recombinase.

22. The method of claim 17, wherein at least one of the first and second selective promoters comprises a tissue specific promoter.

23. The method of claim 17, wherein at least one of the first and second selective promoters comprises a temporally specific promoter.

24. The method of claim 17, wherein at least one of the first and second selective promoters comprises an inducible promoter.

25. The method of claim 17, wherein at least one of the first and second selective promoters comprises a promoter that is selective for neural cells.

26. A method for selective expression of a transgene in a population of cells in an animal, comprising administering to the animal:
a. a first viral vector comprising a nucleic acid encoding a recombinase operably linked to a nucleic acid encoding a first selective promoter;
b. a second viral vector comprising a nucleic acid encoding a transcription factor operably linked to a nucleic acid encoding a second selective promoter, wherein expression of the transcription factor is regulated by expression of the recombinase; and
c. a third viral vector comprising a transgene under the transcriptional control of a response element specific for the transcription factor.

27. The method of claim 26 wherein the first, second and third viral vectors are independently selected from the group consisting of adenovirus vectors, adeno-associated virus vectors, lentivirus vectors, retrovirus vectors and herpesvirus vectors.

28. The method of claim 26 wherein the transgene is a therapeutic molecule, a toxin or a maker protein.

29. The method of claim 28 wherein the therapeutic molecule is a wild-type gene or a pro-apoptotic molecule.

30. The method of claim 26 wherein the animal is a non-human animal.

31. The method of claim 26 wherein the animal is a human.

32. A kit comprising:
a. a first viral vector comprising a nucleic acid encoding a recombinase operably linked to a nucleic acid encoding a first selective promoter;
b. a second viral vector comprising a nucleic acid encoding a transcription factor operably linked to a nucleic acid encoding a second selective promoter; and
c. a third viral vector comprising a transgene under the transcriptional control of a response element specific for the transcription factor.

33. The kit of claim 32, wherein the nucleic acid encoding the transcription factor is flanked by recognition sites for the recombinase.

34. The kit of claim 32, wherein the second viral vector further comprises a transcription STOP signal flanked by recognition sites for the recombinase.

35. A kit comprising:
a. a first injection construct encoding a recombinase operably linked to a first selective promoter;
b. a second injection construct encoding a transcription factor operably linked to a second selective promoter; and
c. a third injection construct comprising a transgene under the transcriptional control of a response element specific for the transcription factor.

36. The kit of claim 35, wherein the transcription factor is flanked by recognition sites for the recombinase.

37. The kit of claim 35, wherein the second injection construct further comprises a transcription STOP signal flanked by recognition sites for the recombinase.
